(19) United States
(12) Patent Application Publication
(10) Pub. No.: US 2023/0114083 A1
(43) Pub. Date: Apr. 13, 2023

(54) PHOTOACOUSTIC IMAGING AGENT

(71) Applicant: National University Corporation Hokkaido University, Sapporo-shi (JP)

(72) Inventors: Mikako OGAWA, Sapporo-shi (JP); Hideo Takakura, Sapporo-shi (JP); Koki Tsuchiya, Sapporo-shi (JP); Tetsuya Taketsugu, Sapporo-shi (JP); Masato Kobayashi, Sapporo-shi (JP)

(21) Appl. No.: 18/050,394

(22) Filed: Oct. 27, 2022

Related U.S. Application Data
(63) Continuation of application No. PCT/JP2022/003297, filed on Jan. 28, 2022.

(30) Foreign Application Priority Data
Jan. 29, 2021 (JP) 2021-013306

(51) Int. Cl. A61K 49/22; C07D 403/08

(52) U.S. Cl. CPC A61K 49/221; C07D 403/08

(57) ABSTRACT

An ICG fluorescence image measured with an excitation light of 740 nm and a fluorescence of 845 nm is shown in FIG. 10, and an ICG fluorescence image measured with an excitation light of 780 nm and a fluorescence of 845 nm is shown in FIG. 11, respectively. As a result, it was observed that the ICG derivative RGD2-PPA-Cy accumulated in the tumor tissue 30 minutes after tail vein administration, regardless of whether the wavelength of the excitation light was 740 nm or 780 nm.

### CROSS-REFERENCE TO RELATED APPLICATIONS

This application is a continuation of International Application No. PCT/JP2022/003297, filed Jan. 28, 2022, which claims priority to Japanese Patent Application No. 2021-013306, filed Jan. 29, 2021, the entire contents of each of which are incorporated herein by reference.

### TECHNICAL FIELD

The present invention relates to an environmentally responsive photoacoustic imaging agent whose photoacoustic signal changes depending on the environment.

### BACKGROUND ART

Photoacoustic imaging (PAI) is a method of detecting and imaging ultrasonic waves (photoacoustic waves) generated by the thermoelastic expansion of substances that absorbed light. This phenomenon in which photoacoustic waves are generated due to the thermoelastic expansion of tissues accompanying the absorption of light energy by fluorescent molecules and tissues is referred to as a photoacoustic effect. Since an acoustic wave is used as a detection signal, imaging of deep parts is possible. For example, sound waves generated by absorption of excitation laser beam can be used to obtain functional images at depths of several centimeters. In addition, since analysis by superimposition with an ultrasonic echo image is also possible, it is expected to be widely used as a useful imaging method for diagnosing pathological conditions in clinical practice in the future. In fact, when a photoacoustic imaging agent targeting prostate-specific membrane antigen (PSMA) was used in prostate cancer model mice, it has been reported that tumors could be detected by overlaying and analyzing photoacoustic images on echo images (see Non-Patent Document 1).

In photoacoustic imaging, although endogenous optical absorbers such as hemoglobin in the living body can be used as optical absorbers for generating photoacoustic waves, studies on exogenous optical absorbers (photoacoustic imaging agents) have been conducted because the range of imaging targets and applications can be broadened. Imaging using a photoacoustic imaging agent is generally carried out by means of accumulation of the imaging agent in the target tissue or target site.

In order to image a deep part of the body, it is preferable to use an optical absorber with an absorption wavelength in the near-infrared region, which is highly permeable to the body, as a photoacoustic imaging agent. For example, indocyanine green (ICG) is an optical absorber having an absorption wavelength in the near-infrared region, and is used for labelling various substances in vivo. For example, an ICG fusion antibody in which a monoclonal antibody is bound directly to ICG having a carboxyl group or via a PEG chain can be used for labelling a target substance in vivo (see, for example, Non-Patent Documents 2 to 4).

### CITATION LIST

### Non-Patent Documents

[Non-Patent Document 1] Zhang et al., Journal of Biophotonics, 2018, vol. 11(9), e201800021.
[Non-Patent Document 2] Zhou et al., Bioconjugate Chemistry, 2014, vol. 25, p. 1801-1810.
[Non-Patent Document 3] Aung et al., Molecular Imaging, 2016, vol. 15, p. 1-[Non-Patent Document 4] Sano et al., Bioconjugate Chemistry, 2013, vol. 24, p. 811-816.

### SUMMARY OF INVENTION

### Technical Problem

A photoacoustic imaging agent always shows a photoacoustic signal in response to the absorption of light of a specific wavelength. Therefore, it is not easy to achieve high contrast imaging due to the influence of non-specific accumulation and the photoacoustic imaging agent remaining in the blood. Further, it is very difficult to target an organ involved in the metabolism and excretion of the photoacoustic imaging agent due to the large background.

An object of the present invention is to provide a compound useful as an active ingredient of an environmentally responsive photoacoustic imaging agent whose optical absorption properties change under a specific environment, and a photoacoustic imaging agent containing this compound as an active ingredient.

### Solution to Problem

The inventors of the present invention have completed the present invention by finding out that optical absorption properties of a derivative in which a specific substituent has been introduced near the center of a methine chain that connects two benzoindolenine rings of ICG change toward the longer wavelength side.

That is, the present invention provides the following ICG derivatives, pharmaceutical compositions, and photoacoustic imaging agents.

[1] An indocyanine green derivative having a structure represented by the following general formulas (1) to (3),

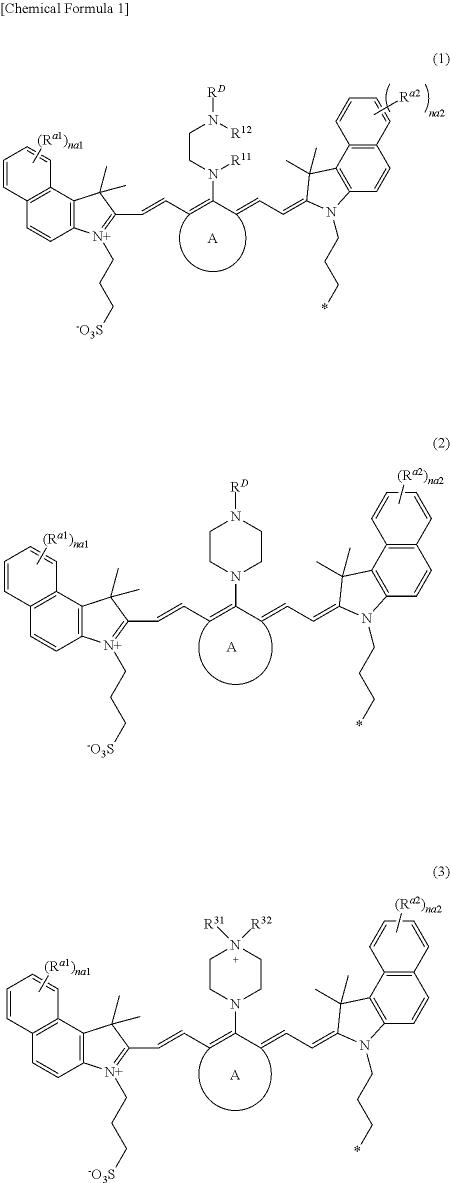

[in the formula, a ring A represents a cyclohexene ring or a cyclopentene ring; R^{D} represents an electron withdrawing group; each of R¹¹ and R¹² independently represents a hydrogen atom or an alkyl group having 1 to 6 carbon atoms which may have a substituent; each of R³¹ and R³² independently represents an alkyl group having 1 to 6 carbon atoms which may have a substituent; each of R^{a1} and R^{a2} independently represents a sulfo group or a carboxy group; each of nₐ₁ and nₐ₂ independently represents 0 or 1; and a filled circle represents a bond.]

[2] The indocyanine green derivative according to the above [1], wherein in the aforementioned general formula (1) or (2), a nitrogen atom bonded to the aforementioned R^{D} has a stronger electron withdrawing property than that of a nitrogen atom bonded to the aforementioned ring A.

[3] The indocyanine green derivative according to the above [1], having a structure represented by the aforementioned general formula (2), wherein

a distance between a nitrogen atom bonded to the aforementioned ring A and a carbon atom in the aforementioned ring A bonded to the nitrogen atom is longer than a distance between a nitrogen atom bonded to a cyclohexene ring in a compound represented by the following formula (Mor-Cy) and a carbon atom in the aforementioned cyclohexene ring bonded to the nitrogen atom, or

an amount of negative charge of the nitrogen atom bonded to the aforementioned ring A is greater than an amount of negative charge of the nitrogen atom bonded to the cyclohexene ring in the compound represented by the following formula (Mor-Cy).

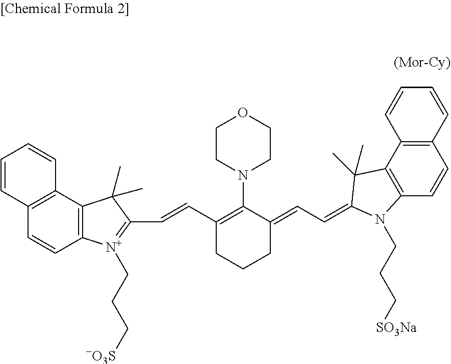

[4] The indocyanine green derivative according to the above [1], having a structure represented by the aforementioned general formula (2), wherein

a distance between a nitrogen atom bonded to the aforementioned ring A and a carbon atom in the aforementioned ring A bonded to the nitrogen atom is longer than a distance between a nitrogen atom bonded to a cyclohexene ring in a compound represented by the following formula (PhP-Cy) and a carbon atom in the aforementioned cyclohexene ring bonded to the nitrogen atom, or an amount of negative charge of the nitrogen atom bonded to the aforementioned ring A is greater than an amount of negative charge of the nitrogen atom bonded to the cyclohexene ring in the compound represented by the following formula (PhP-Cy).

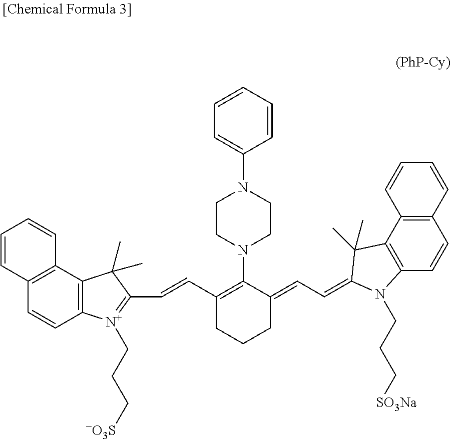

[5] The indocyanine green derivative according to the above [1], having a structure represented by the aforementioned general formula (2), wherein

a distance between a nitrogen atom bonded to the aforementioned ring A and a carbon atom in the aforementioned ring A bonded to the nitrogen atom is 1.375 Å or more.

[6] The indocyanine green derivative according to the above [1], having a structure represented by the aforementioned general formula (2), wherein

an amount of charge of a nitrogen atom bonded to the aforementioned ring A is −0.524 or less.

[7] The indocyanine green derivative according to the above [1], wherein the aforementioned R^{D} is an acyl group, a mesyl group, an aldehyde group, a cyano group, a cyanophenyl group, a nitrophenyl group, or a carboxyalkyl group.

[8] The indocyanine green derivative according to any one of the above [1] to [7], wherein one or more members selected from the group consisting of a protein, a peptide, a nucleic acid, a sugar, a lipid, a polymer and a low molecular weight compound are linked.

[9] The indocyanine green derivative according to the above [8], wherein the aforementioned protein is an antibody or a portion thereof.

[10] A photoacoustic imaging agent containing the indocyanine green derivative according to any one of the above [1] to [9] as an active ingredient.

[11] A pharmaceutical composition containing the indocyanine green derivative according to any one of the above [1] to [9] as an active ingredient.

[12] A method for producing a photoacoustic imaging image, the method including administering the photoacoustic imaging agent according to the above [10] to an individual animal (excluding humans); irradiating near infrared light from the outside, and detecting generated photoacoustic waves to produce a photoacoustic imaging image.

[13] The method for producing a photoacoustic imaging image according to the above [12], further including irradiating the aforementioned individual animal with ultrasonic waves from the outside to produce an echo image, and

superimposing the aforementioned echo image and the aforementioned photoacoustic imaging image.

### Advantageous Effects of Invention

The ICG derivative according to the present invention has optical absorption properties shifted to the long wavelength side. A photoacoustic imaging agent containing this ICG derivative as an active ingredient by utilizing this optical absorption properties with a longer wavelength can reduce the noise of the photoacoustic signal by increasing the wavelength of excitation light to be irradiated, and can detect the target site more accurately. Therefore, the ICG derivative according to the present invention is particularly useful as an active ingredient of a pharmaceutical composition used for obtaining photoacoustic imaging images for analysis of the internal state of an animal body.

### BRIEF DESCRIPTION OF DRAWINGS

FIG. 1 shows absorption spectra of an ICG derivative Mor-Cy measured in Example 1.

FIG. 2 shows absorption spectra of an ICG derivative MP-Cy measured in Example 1.

FIG. 3(A) shows absorption spectra consisted of a relative absorbance of each ICG derivative (aminocyanine) in DMSO in Example 1 (obtained by setting the absorbance of the absorption peak near 700 nm to 1). FIG. 3(B) is a diagram showing the strength of the electron withdrawing properties of the nitrogen-containing groups bonded to the cyclohexene ring of each ICG derivative (aminocyanine).

FIG. 4(A) shows absorption spectra consisted of the relative absorbance of each ICG derivative (aryl piperazine cyanine) in DMSO in Example 1 (obtained by setting the absorbance of the absorption peak near 700 nm to 1). FIG. 4(B) is a diagram showing the strength of the electron withdrawing properties of the nitrogen-containing groups bonded to the cyclohexene ring of each ICG derivative (aryl piperazine cyanine).

FIG. 5 shows absorption spectra of an ICG derivative PBA-Cy measured in Example 2.

FIG. 6 shows photoacoustic imaging images of cells subjected to first irradiation (excitation light of 800 nm) and cells subjected to second irradiation (excitation light of 720 nm) in Example 2.

FIG. 7 shows photoacoustic imaging images of cells subjected to first irradiation (excitation light of 720 nm) and cells subjected to second irradiation (excitation light of 800 nm) in Example 2.

FIG. 8 shows absorption spectra of an ICG derivative PPA-Cy measured in Example 3.

FIG. 9 shows absorption spectra of an ICG derivative SS-PPA-Cy measured in Example 3.

FIG. 10 shows fluorescence imaging images (excitation light 740 nm, fluorescence 845 nm) of mice administered with an ICG derivative PPA-Cy in Example 5.

FIG. 11 shows fluorescence imaging images (excitation light 780 nm, fluorescence 845 nm) of mice administered with an ICG derivative PPA-Cy in Example 5.

### DESCRIPTION OF EMBODIMENTS

ICG is a clinical diagnostic drug that has already been approved by the US FDA, and is a near-infrared optical absorber known to have extremely low toxicity to living organisms. ICG has a high molar extinction coefficient and easily generates photoacoustic waves, but has a problem of low photostability. In addition, since ICG is not environmentally responsive, when ICG is used as a photoacoustic imaging agent, photoacoustic waves are also generated from ICG present outside the target site, and noise level is high in the acquired photoacoustic imaging image, and accuracy tends to be insufficient.

On the other hand, the ICG derivative according to the present invention has different optical absorption properties from those of the ICG derivative in which no substituent is introduced into the methine chain connecting the benzindole rings at both ends. More specifically, the maximum absorption wavelength is shifted to the longer wavelength side. By irradiating the ICG derivative with light, a photoacoustic signal corresponding to the absorption spectrum is detected. That is, the ICG derivative according to the present invention is suitable as an active ingredient of a photoacoustic imaging agent because noise can be suppressed by irradiating light with a longer wavelength as compared with the related art and detecting a photoacoustic signal.

The ICG derivative according to the present invention is a compound having a structure represented by any one of the following general formulas (1) to (3). It should be noted that in the general formulas (1) to (3), filled circles represent bonds.

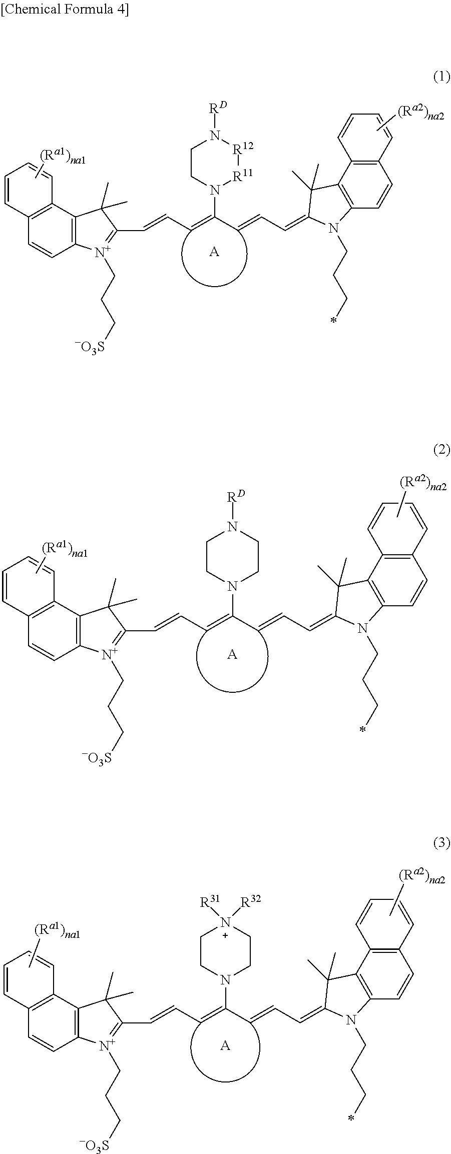

In the general formulas (1) to (3), a ring A represents a cyclohexene ring or a cyclopentene ring. By introducing a 6- or 5-membered alicyclic structure near the center of the methine chain that connects the two benzoindolenine rings of ICG, the ICG derivative according to the present invention is more photostable than ICG. As for the ICG derivative according to the present invention, the ring A is preferably a cyclohexene ring.

In the general formula (1), each of R¹¹ and R¹² independently represents a hydrogen atom or an alkyl group of 1 to 6 carbon atoms which may have a substituent. This alkyl group having 1 to 6 carbon atoms may be a linear group or a branched group. Examples of the alkyl group having 1 to 6 carbon atoms include a methyl group, an ethyl group, a propyl group, an isopropyl group, a butyl group, an isobutyl group, a sec-butyl group, a tert-butyl group, a pentyl group, an isopentyl group, a neopentyl group, a tert-pentyl group and a hexyl group.

When R¹¹ and R¹² are alkyl groups having 1 to 6 carbon atoms, these alkyl groups may have 1 to 3 substituents. The substituent is not particularly limited as long as it does not impair the effects of the present invention, and examples thereof include a phenyl group which may have 1 to 3 substituents. Examples of the substituents included in the phenyl group include alkyl groups having 1 to 6 carbon atoms, carboxyalkyl groups, and the like.

In addition, the substituents included in the alkyl group may be groups composed of substances such as peptides, proteins, low molecular weight compounds, sugars, nucleic acids, lipids and polymers, or those obtained by binding these groups directly or via an appropriate linking group. Examples of the linking group include an alkylene group, an alkenylene group, a carbonyl group (—CO—), an ether bond (—O—), an ester bond (—COO—), an amide bond (—CONH—) and polyethylene glycol (PEG: —(C₂H₄O)n-), and these can also be used in appropriate combination.

Examples of peptides and proteins include among the substances contained in the substituents of the above alkyl groups include enzymes, antibodies or portions thereof, antigens, peptide tags, fluorescent proteins, ligands and peptide aptamers for various biomolecules, and agonists, antagonists and peptide drugs for various receptors. The nucleic acid may be a natural nucleic acid such as DNA or RNA, or an artificial nucleic acid. The nucleic acid is preferably a functional nucleic acid such as a nucleic acid medicine or a nucleic acid aptamer. The sugar may be a monosaccharide, a disaccharide, an oligosaccharide, or a sugar chain (polysaccharide) such as a glycosaminoglycan. Examples of the lipid include glycerophospholipids, sphingophospholipids, sterols and fatty acids. The lipid is also preferably a constituent lipid of lipid nanoparticles. Lipid nanoparticles to which an ICG derivative is linked can be obtained by using an ICG derivative linked to a lipid as a constituent lipid. A polymer is a macromolecule obtained by polymerizing a large number of monomers, and in the present invention, proteins, nucleic acids, and sugar chains are excluded. Examples of the polymer include polyalkylene glycols such as polyethylene glycol and polypropylene glycol; polyalkylene succinates such as polybutylene succinate; and polycarboxylic acids such as polylactic acid. As the low molecular weight compound, fluorescent materials and medicinal ingredients are preferred.

In the general formula (3), each of R³¹ and R³² independently represents an alkyl group of 1 to 6 carbon atoms which may have a substituent. As the alkyl group of 1 to 6 carbon atoms represented by R³¹ and R³² which may have a substituent, the same groups as those mentioned for R¹¹ and R¹² can be used.

The maximum absorption wavelength of the ICG derivative having the structure represented by the general formula (3) shifts to the longer wavelength side than that of an ICG derivative having no substituent on the ring A or an ICG derivative into which an electron donating group has been introduced on the nitrogen atom at the 4th position of the piperazine ring bonded to the ring A. Although the mechanism of such change in wavelength has not been elucidated yet, in the ICG derivative having the structure represented by the general formula (3), the nitrogen atom at the 4th position of the piperazine ring bonded to the ring A is a monovalent cation and has a stronger electron withdrawing property than that of the nitrogen atom at the 1st position in the piperazine ring (the nitrogen atom bonded to the ring A). Therefore, it is presumed that in the ICG derivative having the structure represented by the general formula (3), a conjugated system of the methine chain that connects the benzindole rings at both ends is not divided by the ring A and the piperazine ring bonded thereto, so that light of a long wavelength can be absorbed.

In the general formulas (1) and (2), R^{D} represents an electron withdrawing group. An ICG derivative having a structure represented by the general formula (1) or (2) has nitrogen-containing groups having nitrogen atoms are bound to the ring A at the 1st position and 4th position, and the nitrogen atom at the 4th position is bound to an electron withdrawing group. Since the nitrogen atom at the 4th position (the nitrogen atom to which R^{D} is bound) has a greater electron withdrawing property than that of the nitrogen atom at the 1st position (the nitrogen atom to which the ring A is bound) due to this structure, the maximum absorption wavelength shifts to the longer wavelength side, similar to the case of the ICG derivative having the structure represented by the general formula (3).

The electron withdrawing group represented by R^{D} is not particularly limited as long as it is a group capable of enhancing the electron withdrawing properties of the nitrogen atom at the 4th position so that the maximum absorption wavelength of the ICG derivative having the structure represented by the general formula (1) or (2) can be shifted to the longer wavelength side. Examples of the electron withdrawing group include an acyl group, a mesyl group, an aldehyde group, a cyano group, an alkyl group substituted with a highly electronegative functional group, and an aryl group substituted with a highly electronegative functional group. An acyl group, a mesyl group, an aldehyde group, and an alkyl group portion in an alkyl group substituted with a highly electronegative functional group are not particularly limited, and the same as those listed for R¹¹ and R¹² can be used. Examples of the aryl group substituted with a highly electronegative functional group include a cyanophenyl group, a nitrophenyl group, a cyanonaphthyl group and a nitronaphthyl group. The alkyl group substituted with a highly electronegative functional group is preferably a sulfoalkyl group (group in which one hydrogen atom of an alkyl group is substituted with a sulfo group (—SO₃H)) or a carboxyalkyl group (group in which one hydrogen atom of an alkyl group is substituted with a carboxy group), more preferably a sulfoalkyl group having 1 to 6 carbon atoms or a carboxyalkyl group having 1 to 6 carbon atoms, and still more preferably a sulfoalkyl group having 1 to 3 carbon atoms or a carboxyalkyl group having 1 to 3 carbon atoms.

The electron withdrawing group represented by R^{D} may be a group to which peptides, proteins, low molecular weight compounds, sugars, nucleic acids, lipids, polymers and the like are bonded directly or via an appropriate linking group. For any of the peptides, proteins, low molecular weight compounds, sugars, nucleic acids, lipids, polymers and linking groups included in R^{D}, when R¹¹ and R¹² are alkyl groups having 1 to 6 carbon atoms, those listed as the substituents these alkyl groups may have can be used.

In the general formulas (1) to (3), each of R^{a1} and R^{a2} independently represents a sulfo group or a carboxy group. Further, in the general formulas (1) to (3), each of nₐ₁ and nₐ₂ independently represents 0 or 1. The ICG derivative according to the present invention is preferably a compound having any one of the structures represented by the following general formulas (1-1) to (1-2), (2-1) to (2-2) and (3-1) to (3-2) or a salt thereof. Examples of the salt include sodium salts and potassium salts.

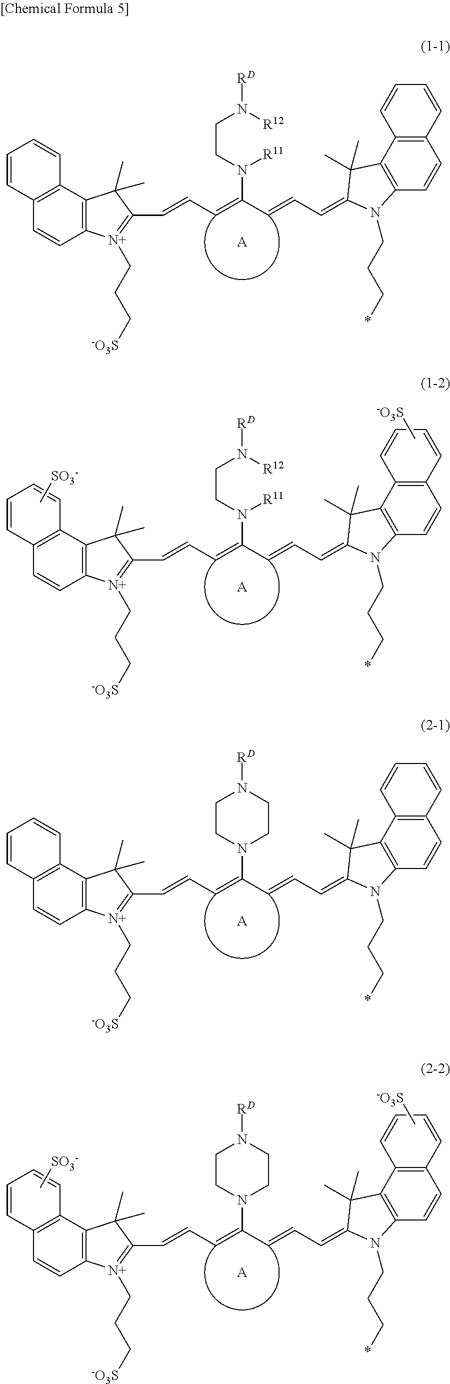

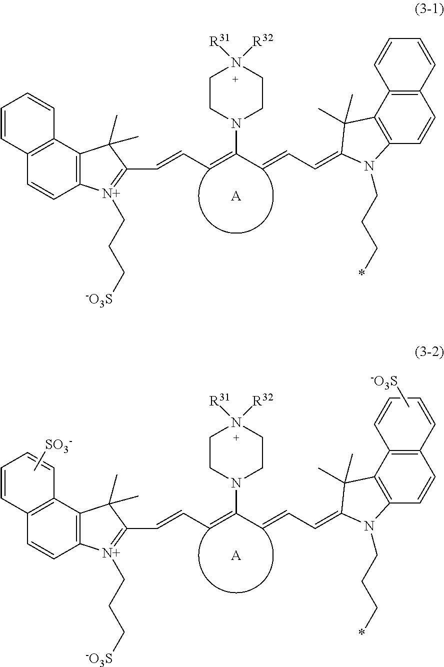

In the ICG derivative according to the present invention, in the nitrogen-containing group bonded to the ring A, there is a tendency that the stronger the electron withdrawing property of the 4th position nitrogen atom than that of the 1st position nitrogen atom, the greater the difference in maximum absorption wavelength. Since the shift of the maximum absorption wavelength to the long wavelength side can be further increased, the longer the distance (Å) between the nitrogen atom at the 1st position (nitrogen atom bonded to the ring A) and the carbon atom in the ring A bonded to the nitrogen atom in the ICG derivative according to the present invention, the better. In addition, it is preferable that the amount of negative charge of the nitrogen atom bonded to the ring A is larger. It should be noted that in the following description, the “distance between the nitrogen atom at the 1st position (nitrogen atom bonded to the ring A) and the carbon atom in the ring A bonded to the nitrogen atom” in the ICG derivative may be referred to as “L (C—N)”.

For example, in the case of an ICG derivative having a structure represented by the general formula (2), L(C—N) is preferably longer than L(C—N) in the ICG derivative Mor-Cy described later (distance between the nitrogen atom bonded to the cyclohexene ring and the carbon atom in the cyclohexene ring bonded to this nitrogen atom). In addition, in the case of an ICG derivative having a structure represented by the general formula (2), L(C—N) is also preferably longer than L(C—N) in the ICG derivative PhP-Cy described later (distance between the nitrogen atom bonded to the cyclohexene ring and the carbon atom in the cyclohexene ring bonded to this nitrogen atom). In the case of the ICG derivative having the structure represented the by general formula (2), L(C—N) is also preferably 1.375 Å or longer.

Further, in the case of the ICG derivative having the structure represented by the general formula (2), the amount of negative charge of the nitrogen atom bonded to the ring A is preferably larger than the amount of negative charge of the nitrogen atom bonded to the cyclohexene ring in the ICG derivative Mor-Cy. Moreover, in the case of the ICG derivative having the structure represented by the general formula (2), the amount of negative charge of the nitrogen atom bonded to the ring A is also preferably larger than the amount of negative charge of the nitrogen atom bonded to the cyclohexene ring in the ICG derivative PhP-Cy. In the case of the ICG derivative having the structure represented by the general formula (2), the amount of charge of the nitrogen atom bonded to the ring A is also preferably −0.524 or less, and more preferably −0.53 or less.

L(C—N) in the ICG derivative can be obtained by using, for example, a molecular mechanics calculation program using a quantum chemical calculation program. As the quantum chemistry calculation program, for example, a widely used quantum chemistry calculation program such as “Gaussian 16 program” (manufactured by Gaussian, Inc.) can be used. As a molecular mechanics program using a quantum chemical calculation program, for example, a widely used molecular mechanics calculation program such as “CONFLEX (registered trademark) 8 program” (manufactured by CONFLEX Corporation) can be used. For example, using a molecular mechanics calculation program that utilizes a quantum chemistry calculation program, first, a conformational search for each ICG derivative is performed, and the most stable conformational structure is specified by structural optimization calculation. L(C—N) measured from this stable conformational structure is defined as L(C—N) of this ICG derivative. In addition, the amount of negative charge of the nitrogen atom bonded to the ring A in the obtained stable conformational structure can be calculated using natural population analysis, and the calculated charge amount is taken as the amount of negative charge of the nitrogen atom bonded to the ring A of this ICG derivative.

By using a molecular mechanics calculation program that utilizes this quantum chemical calculation program, it is also possible to screen for ICG derivatives that have a greater shift of the maximum absorption wavelength to the longer wavelength side and are capable of detecting excitation light signals of longer wavelengths. For example, for a compound library containing ICG derivatives, each L(C—N) and the amount of negative charge of the nitrogen atom bonded to the ring A are calculated. An ICG derivative having a longer L(C—N) and a larger amount of negative charge of the nitrogen atom bonded to the ring A is selected as an ICG derivative having a large shift of the maximum absorption wavelength to the longer wavelength side. An ICG derivative with a large shift of the maximum absorption wavelength to the long wavelength side is particularly suitable as an in vivo photoacoustic imaging agent, and it is possible to select an ICG derivative capable of producing a photoacoustic imaging image with less noise and a high S/N ratio by this screening method.

In addition, by using a molecular mechanics calculation program using this quantum chemical calculation program for a candidate ICG derivative of a photoacoustic imaging agent, it is also possible to predict the easiness of shifting the maximum absorption wavelength of this candidate ICG derivative to the longer wavelength side. For example, the amount of negative charge of the nitrogen atom that binds to L(C—N) and ring A of the candidate ICG derivative is calculated. When the calculated L(C—N) value is longer than a preset reference value, it is predicted that the maximum absorption wavelength of the candidate ICG derivative is likely to shift to the long wavelength side. In addition, when the calculated value of the amount of negative charge of the nitrogen atom bonded to the ring A is larger than a preset reference value, it is predicted that the maximum absorption wavelength of the candidate ICG derivative is likely to shift to the long wavelength side. The L(C—N) value or the value of the amount of negative charge of the nitrogen atom bonded to the ring A of the ICG derivative whose maximum absorption wavelength has been measured in advance can be used as the reference value.

In the ICG derivative according to the present invention, a structural portion other than the structural portion represented by the aforementioned general formulas (1) to (3) is not particularly limited as long as it does not impair the effects as a photoacoustic imaging agent due to the structure represented by the aforementioned general formulas (1) to (3). Examples of the structure of a portion to which the bond in the aforementioned general formulas (1) to (3) binds include, for example, the same sulfo group as in ICG or a salt thereof, and a sulfo group bound to any linking group or a salt thereof. Examples of the linking group include the same as those described above.

In the ICG derivative according to the present invention, the structural portion to which the bonds in the general formulas (1) to (3) are bonded may have the same structure as that of a known ICG derivative. Examples of the known ICG derivative include an ICG derivative in which a bond is bound to a labeling material such as a fluorescent material directly or via an arbitrary linking group. Examples of the known ICG derivative in which the bond is bound to a group containing a fluorescent material include ICG-Sulfo-OSu (code: 1254, manufactured by Dojindo Laboratories), ICG-EG4-Sulfo-OSu (code: 1289, manufactured by Dojindo Laboratories), and ICG-EG8-Sulfo-OSu (code: 1290, manufactured by Dojindo Laboratories).

When the ICG derivative according to the present invention is used as an active ingredient of a photoacoustic imaging agent, it can have a structure in which a molecule capable of binding to a target molecule to be detected by the photoacoustic imaging agent is bound to an arbitrary linking group. Examples of the linking group include the same as those described above. The molecule capable of binding to a target molecule may be a peptide or protein, a nucleic acid, a lipid, a sugar or a sugar chain, or a low molecular weight compound.

For example, when the ICG derivative according to the present invention is an ICG derivative having a structure represented by the aforementioned general formula (1), an ICG derivative to which a target molecule is linked can be obtained by allowing R¹¹ or R¹² to be, a group obtained by substituting at least one hydrogen atom in an alkyl group having 1 to 6 carbon atoms with a group composed of a molecule capable of binding to the target molecule, or a group in which this group is bound to a linking group. When the ICG derivative according to the present invention is an ICG derivative having a structure represented by the aforementioned general formula (1) or (2), an ICG derivative to which a target molecule is linked can be obtained by allowing R^{D} to be a group in which the target molecule is bound to the electron withdrawing group directly or through an appropriate linking group. When the ICG derivative according to the present invention is an ICG derivative having a structure represented by the aforementioned general formula (3), an ICG derivative to which a target molecule is linked can be obtained by allowing R³¹ or R³² to be a group obtained by substituting at least one hydrogen atom in an alkyl group having 1 to 6 carbon atoms with a group composed of a molecule capable of binding to the target molecule, or a group in which this group is bound to a linking group.

Examples of molecules capable of binding to target molecules include antibodies and ligands for the target molecules. For example, by using a biomolecule present on the surface of a tumor tissue or tumor cell as a target molecule and using an ICG derivative containing a molecule capable of binding to the target molecule as an active ingredient of a photoacoustic imaging agent, a detectable photoacoustic imaging agent capable of detecting tumors by photoacoustic imaging can be obtained.

In general, expression of integrins is often seen in tumor cells. Accordingly, when the target molecule is a tumor cell, an integrin-binding peptide containing an RGD sequence can be used as a molecule capable of binding to the target molecule. By using an ICG derivative in which an integrin-binding peptide and a structure represented by any of the aforementioned general formulas (1) to (3) are bound directly or via an appropriate linking group, or an ICG derivative containing an integrin-binding peptide in a structure represented by any of the aforementioned general formulas (1) to (3) as a photoacoustic imaging agent, a photoacoustic imaging image of tumor cells in the body of an animal can be obtained.

Since the ICG derivative according to the present invention has ICG as its basic skeleton, it can be expected to have low toxicity to living organisms like ICG. Moreover, the photoacoustic wave generated from the ICG derivative according to the present invention is a sound wave with high biopermeability. Therefore, the ICG derivative is also suitable as an active ingredient of a pharmaceutical composition to be administered to animals including humans.

Pharmaceutical compositions containing the ICG derivative according to the present invention as an active ingredient can be formulated by ordinary methods into oral solid formulations such as powders, granules, capsules, tablets, chewable formulations and sustained release formulations, oral liquid formulation such as solution formulations and syrups, injections, enema agents, sprays, patches, ointments, and the like. Formulation can be carried out by a conventional method by blending, if necessary for the formulation, excipients, binders, lubricants, disintegrants, fluidizers, solvents, solubilizers, buffering agents, suspending agents, emulsifiers, tonicity agents, stabilizers, preservatives, antioxidants, flavoring agents, coloring agents and the like.

The ICG derivative according to the present invention has optical absorption properties shifted to the long wavelength side. Therefore, the ICG derivative according to the present invention is particularly suitable as an active ingredient of a photoacoustic imaging agent for detecting molecules and tissues in vivo.

The route of administration of the photoacoustic imaging agent and pharmaceutical composition containing the ICG derivative according to the present invention as an active ingredient is not particularly limited, and is appropriately determined depending on the target cells and tissues containing them. Examples of the route of administration of the photoacoustic imaging agent and the like containing the ICG derivative according to the present invention as an active ingredient include oral administration, intravenous administration, intraperitoneal administration and enema administration.

The animal to which the photoacoustic imaging agent and pharmaceutical composition containing the ICG derivative according to the present invention as an active ingredient is administered is not particularly limited, and may be a human or a non-human animal. Examples of the non-human animal include mammals such as cattle, pigs, horses, sheep, goats, monkeys, dogs, cats, rabbits, mice, rats, hamsters and guinea pigs, and birds such as chickens, quails and ducks.

The photoacoustic imaging agent containing the ICG derivative according to the present invention as an active ingredient can be used in the same manner as the photoacoustic imaging agents used in the production of conventional photoacoustic imaging images. More specifically, first, the photoacoustic imaging agent according to the present invention is administered to an individual animal. Next, this individual animal is irradiated with near infrared light from the outside, and the generated photoacoustic wave signal is detected. Detection of the photoacoustic signal can be performed using an ultrasonic detector used in echography or the like, and a photoacoustic imaging image can be produced from the detected photoacoustic signal by a conventional method.

Although the wavelength of the near infrared light to be irradiated is not particularly limited as long as it is a wavelength at which the photoacoustic imaging agent can generate photoacoustic waves, a wavelength near the maximum absorption wavelength of the photoacoustic imaging agent is preferred because a photoacoustic imaging image with less noise and a high S/N ratio can be produced. For example, by irradiating near infrared light having a wavelength of 800 nm or more, a photoacoustic imaging image with less noise and a high S/N ratio can be produced.

The photoacoustic imaging agent containing the ICG derivative according to the present invention as an active ingredient has optical absorption properties shifted to the longer wavelength side when it is incorporated into cells than that when it is not incorporated into cells. By utilizing this difference in optical absorption properties and irradiating light with a longer wavelength, for example, near infrared light having a wavelength of 800 nm or more, it is possible to suppress the adverse effects of noise on the photoacoustic signal generated and obtained from the ICG derivative while being incorporated into cells.

Furthermore, echography can also be performed on an individual animal that has been administered with a photoacoustic imaging agent. More specifically, this individual animal is irradiated with ultrasonic waves from the outside to create an echo image. By superimposing and analyzing the obtained echo image and photoacoustic imaging image, the target cell can be analyzed in more detail.

### EXAMPLES

Next, the present invention will be described in more detail with reference to Examples, but the present invention is not limited to the following Examples.

### Example 1

An ICG derivative was synthesized and its optical absorption spectrum was measured.

### Synthesis of ICG Derivative

Ten types of ICG derivatives were synthesized by the following synthesis reactions. The synthesized ICG derivative was analyzed by NMR and MS to confirm that an ICG derivative having the desired structure was synthesized.

### (1) Synthesis of 1,1,2-trimethyl-3-(3-sulfopropyl)-1H-benz[e]indolium (Compound 1)

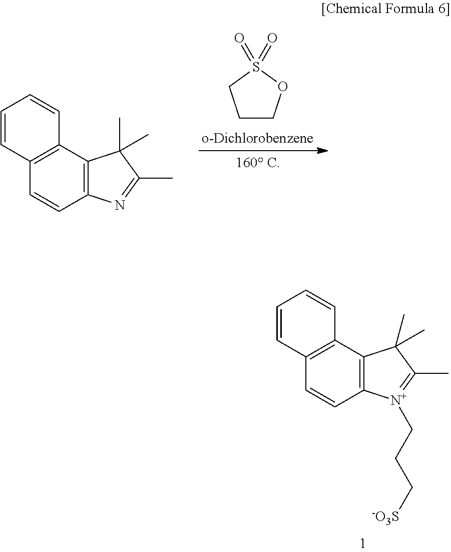

2,3,3-trimethyl-3H-indole (2.0 g, 9.56 mmol) and propane sultone (1.3 g, 10.6 mmol) were dissolved in anhydrous o-dichlorobenzene (5 mL). This mixed solution was subjected to argon substitution and stirred at 160° C. for 2 hours, and the progress of the reaction was confirmed by TLC. This reaction solution was subjected to suction filtration using cold hexane, and the precipitated solid was dried in vacuo to obtain a compound 1 as a gray solid (3.0 g, 95% yield).

¹H-NMR (400 MHz, DMSO-D6): δ 8.35 (d, 1H, J=8.5 Hz), 8.28 (d, 1H, J=9.0 Hz), 8.24-8.18 (m, 2H), 7.77 (t, 1H, J=7.2 Hz), 7.71 (t, 1H, J=7.4 Hz), 4.76 (t, 2H, J=7.9 Hz), 2.92 (s, 3H), 2.65 (t, 2H, J=6.5 Hz), 2.25-2.16 (m, 2H), 1.74 (s, 6H)

### (2) Synthesis of N-[[2-chloro-3-[(phenylamino)methylene]-1-cyclohexen-1-yl]methylene]benzenamine (compound 2)

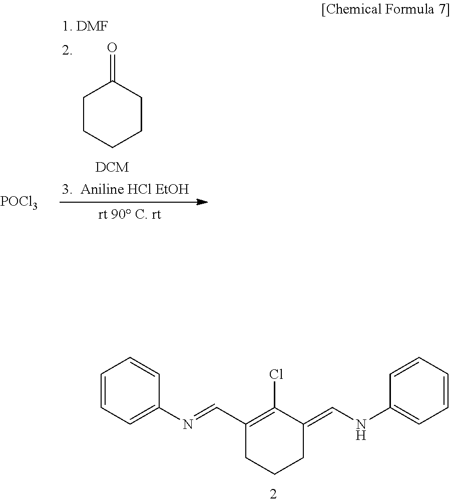 [SMILES: O=C1CCCCC1 ; "POCl3" ; ClC1=C(/C=N/c2ccccc2)CCC/C1=C\Nc1ccccc1]

Phosphoryl chloride (2.5 mL, 26.8 mmol) was added dropwise to anhydrous N,N-dimethylformamide (DMF) (3.0 mL, 38.7 mmol) under ice cooling, and the resulting mixture was allowed to return to room temperature and stirred for 1 hour. Cyclohexanone: dichloromethane (volume ratio 1:1, 1.5 mL each, cyclohexanone 14.5 mmol) were added to this solution and stirred at 100° C. for 2 hours. After adding ethanol (5 mL) to this solution, aniline: ethanol (volume ratio 1:1, 5 mL each, aniline 54.9 mmol) were added dropwise under ice cooling, and the resulting mixture was allowed to return to room temperature and stirred for 2 hours. The obtained dark red solution was poured into a beaker containing 1.5 M hydrochloric acid (200 mL) to confirm the formation of a precipitate. This solution was subjected to suction filtration using cold H₂O and cold hexane, and the obtained solid was dried in vacuo to obtain a compound 2 as a dark red crude product (5.084 g). This crude product was used for subsequent cyanine synthesis without further processing other than the above purification.

LRMS (ESI⁺): calcd. for [M+H⁺] 323.13, found: 323.35.

### (3) Synthesis of 2-[2-[3-[2-[1,3-dihydro-3,3-dimethyl-1-(3-sulfopropyl)-2H-benz[e]indol-2-ylidene]ethylidene]-2-chloro-1-cyclohexen-1-yl]ethenyl]-3,3-dimethyl-1-(3-sulfopropyl)-3H-benz[e]indolium, Inner Salt, Sodium Salt (Compound 3)

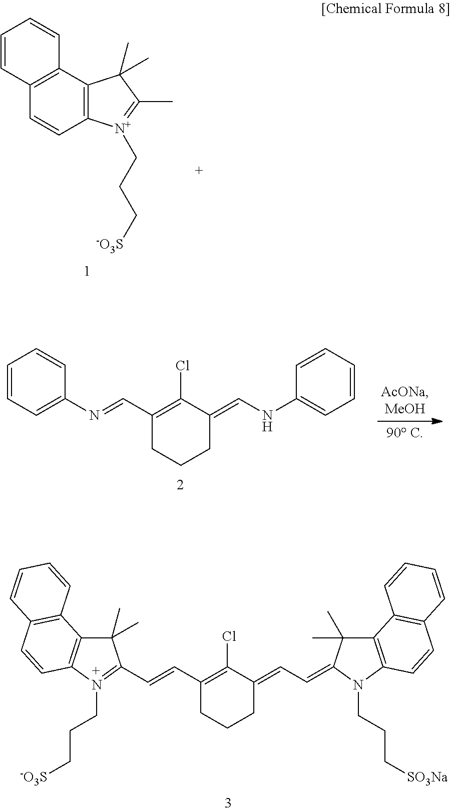

The compound 1 (663.0 mg, 2.0 mmol), the compound 2 (323.0 mg, 1.0 mmol), and anhydrous sodium acetate (246.0 mg, 3.0 mmol) were dissolved in anhydrous methanol (12 mL), and after argon substitution, stirred under reflux at 70° C. for 1.5 hours. After confirming the progress of the reaction by HPLC, the solvent was removed under reduced pressure. The obtained crude product was dissolved in methanol (50 mL) and purified by medium pressure preparative chromatography. After removing triethylamine from the obtained solution and passing the resultant through a cation exchange resin, the solvent was removed under reduced pressure to prepare a solution in which a compound 3 as the desired product was dissolved in water. This solution was purified by freeze drying to obtain a shiny orange solid (223.4 mg, 27.2% yield).

¹H-NMR (400 MHz, CD₃OD): δ 8.56 (d, 2H, J=14.4 Hz), 8.27 (d, 2H, J=8.5 Hz), 8.05-7.96 (m, 4H), 7.73 (d, 2H, J=8.5 Hz), 7.64 (t, 2H, J=7.2 Hz), 7.48 (t, 2H, J=7.2 Hz), 6.50 (d, 2H, J=14.4 Hz), 4.51 (t, 4H, J=7.9 Hz), 3.01 (t, 4H, J=6.7 Hz), 2.82 (t, 4H, J=5.8 Hz), 2.37-2.27 (m, 4H), 2.04 (s, 12H), 2.01-1.94 (m, 2H)

HRMS (ESI⁻): calcd. for [M-Na⁺] 797.24913, found: 797.25139.

### (4) Synthesis of 4-boc-1,1-dimethylpiperazium (Compound 5) and 1,1-dimethylpiperazium (Compound 6)

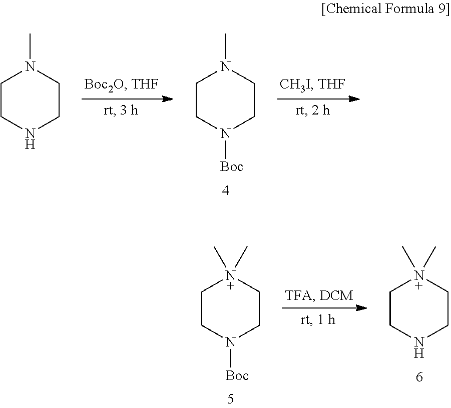

1-methylpiperazine (0.4 mL, 3.6 mmol) and Boc₂O (0.96 g, 4.4 mmol) were dissolved in tetrahydrofuran (THF) (5 mL), and the resulting solution was stirred at room temperature for 3 hours. Formation of a compound 4 as the desired product was confirmed by LRMS, and this solution was used as it is for the next reaction.

LRMS (ESI⁺): calcd. for [M+H⁺] 200.15, found: 201.33.

Iodomethane (0.2 mL, 3.2 mmol) was added to the solution in which the compound 4 was dissolved, and the resulting mixture was stirred at room temperature for 2 hours. The solution after the reaction was filtered under reduced pressure using cold Et₂O. The obtained precipitate was dried in vacuo to obtain the desired compound 5 as a white solid (856.5 mg, theoretical yield).

¹H-NMR (400 MHz, CDCl₃): δ 3.87-3.72 (m, 8H), 3.66 (s, 6H), 1.49 (s, 9H) HRMS (ESI⁺): calcd. for [M+H⁺] 215.17540, found: 215.17545.

### (5) Synthesis of ICG Derivative (Aminocyanine)

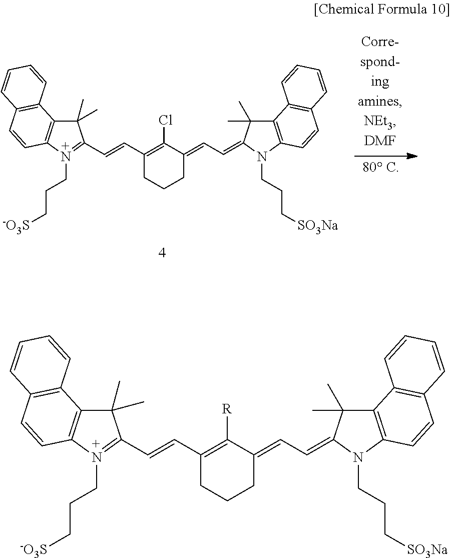

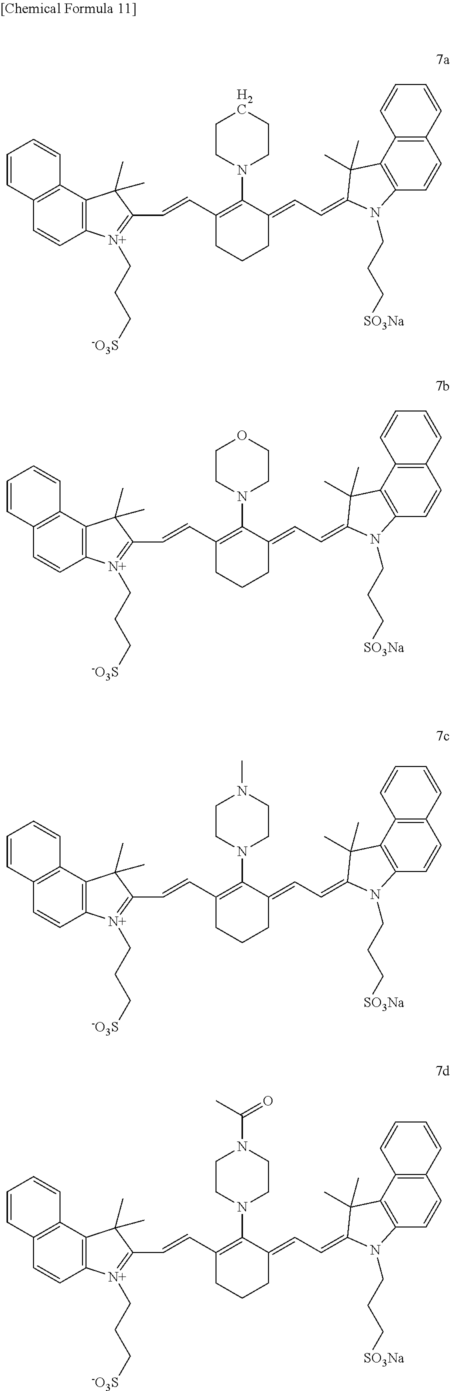

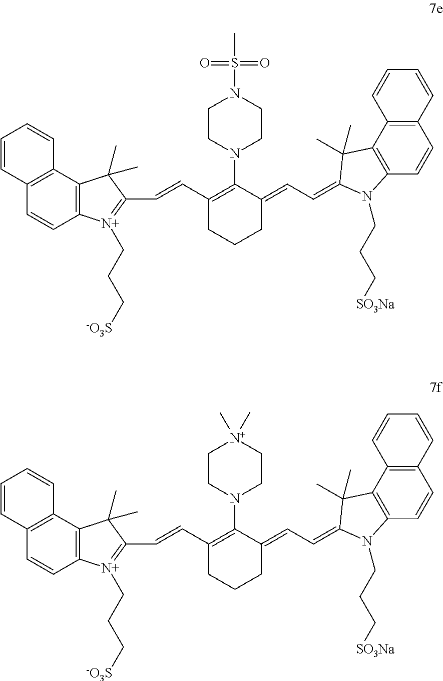

The compound 4 (1 eq.) and the corresponding amine (3 to 14 eq.) were dissolved in anhydrous DMF (0.04 to 0.1 mL/μmol), and after adding triethylamine (0 to 123 eq.) thereto and performing argon substitution, the resulting mixture was stirred at 80° C. After confirming the progress of the reaction by HPLC (ODS silica gel, eluent A: 20 mM triethylamine, eluent B: MeCN/1% H₂O), the solvent was removed under reduced pressure, and the product was purified by preparative HPLC. The obtained solution was passed through a cation exchange resin, and the solvent was removed under reduced pressure to prepare a solution in which an ICG derivative as the desired product was dissolved in water. This solution was freeze dried to obtain a solid of the target ICG derivative.

The raw materials and yields of each compound are shown below.

### (5-1) Synthesis of 2-[2-[3-[2-[1,3-dihydro-3,3-dimethyl-1-(3-sulfopropyl)-2H-benz[e]indol-2-ylidene]ethylidene]-2-(1-piperidinyl)-1-cyclohexen-1-yl]ethenyl]-3,3-dimethyl-1-(3-sulfopropyl)-3H-benz[e]indolium, Inner Salt, Sodium Salt (Compound 7a: Piperi-Cy)

The compound 4 (62 mg, 60.9 μmol), piperidine (18.7 μL, 189.3 μmol) and triethylamine (60.0 μL, 430.5 μmol) were dissolved in anhydrous DMF (7 mL), and synthesis was conducted according to the procedure described above to obtain a compound 7a as a red purple solid (22.2 mg, 42% yield).

¹H-NMR (400 MHz, CD₃OD): δ 8.18 (d, 2H, J=8.1 Hz), 7.94-7.89 (m, 4H), 7.81 (d, 2H, J=13.9 Hz), 7.59-7.52 (m, 4H), 7.38 (t, 2H, J=7.4 Hz), 6.09 (d, 2H, J=12.1 Hz), 4.34-4.26 (m, 4H), 3.92-3.83 (m, 4H), 2.98 (t, 4H, J=7.0 Hz), 2.61 (t, 4H, J=6.7 Hz), 2.31-2.21 (m, 4H), 2.03-1.94 (m, 18H), 1.91-1.83 (m, 2H)

HRMS (ESI⁻): calcd. for [M-Na⁺] 846.36160, found: 846.36404.

### (5-2) Synthesis of 2-[2-[3-[2-[1,3-dihydro-3,3-dimethyl-1-(3-sulfopropyl)-2H-benz[e]indol-2-ylidene]ethylidene]-2-(4-morpholinyl)-1-cyclohexen-1-yl]ethenyl]-3,3-dimethyl-1-(3-sulfopropyl)-3H-benz[e]indolium, Inner Salt, Sodium Salt (Compound 7b: Mor-Cy)

The compound 4 (62.3 mg, 71.1 μmol) and morpholine (60 mg, 688.7 μmol) were dissolved in anhydrous DMF (3 mL), and synthesis was conducted according to the procedure described above to obtain a compound 7b as a red purple solid (18.4 mg, 30% yield).

¹H-NMR (400 MHz, CD₃OD): δ 8.22 (d, 2H, J=8.5 Hz), 8.02-7.91 (m, 6H), 7.64-7.55 (m, 4H), 7.41 (t, 2H, J=7.6 Hz), 6.20 (d, 2H, J=13.5 Hz), 4.36 (t, 4H, J=7.6 Hz), 4.07-4.01 (m, 4H), 3.81-3.76 (m, 4H), 2.99 (t, 4H, J=7.0 Hz), 2.63 (t, 4H, J=6.5 Hz), 2.33-2.23 (m, 4H), 2.02 (s, 12H), 1.91-1.84 (m, 2H) HRMS (ESI⁻): calcd. for [M-Na⁺] 848.34087, found: 846.34180.

### (5-3) Synthesis of 2-[2-[3-[2-[1,3-dihydro-3,3-dimethyl-1-(3-sulfopropyl)-2H-benz[e]indol-2-ylidene]ethylidene]-2-(4-methyl-1-piperazinyl)-1-cyclohexen-1-yl]ethenyl]-3,3-dimethyl-1-(3-sulfopropyl)-3H-benz[e]indolium, Inner Salt, Sodium Salt (Compound 7c: MP-Cy)

The compound 4 (60.2 mg, 73.3 μmol) and 1-methylpiperazine (100.0 mg, 1.0 mmol) were dissolved in anhydrous DMF (3 mL), and synthesis was conducted according to the procedure described above to obtain a compound 7c as a red purple solid (19.8 mg, 30% yield).

¹H-NMR (400 MHz, DMSO-D6): δ8.24 (d, 2H, J=8.5 Hz), 8.02-7.97 (m, 4H), 7.82 (d, 2H, J=13.5 Hz), 7.72 (d, 2H, J=9.0 Hz), 7.58 (t, 2H, J=7.6 Hz), 7.42 (t, 2H, J=7.6 Hz), 6.16 (d, 2H, J=14.8 Hz), 4.32 (t, 4H, J=7.2 Hz), 3.74-3.68 (m, 4H), 3.32-3.28 (m, 4H), 2.57 (t, 8H, J=6.7 Hz), 2.41 (s, 3H), 2.01 (t, 4H, J=7.4 Hz), 1.93 (s, 12H), 1.76 (t, 2H, J=6.7 Hz)

HRMS (ESI⁻): calcd. for [M-Na⁺] 861.37250, found: 861.37419.

### (5-4) Synthesis of 2-[2-[3-[2-[1,3-Dihydro-3,3-dimethyl-1-(3-sulfopropyl)-2H-benz[e]indol-2-ylidene]ethylidene]-2-(4-acetyl-1-piperazinyl)-1-cyclohexen-1-yl]ethenyl]-3,3-dimethyl-1-(3-sulfopropyl)-3H-benz[e]indolium, Inner Salt, Sodium Salt (Compound 7d: AcP-Cy)

The compound 4 (50 mg, 60.9 μmol), 1-acetylpiperazine (21.5 μL, 180.4 μmol) and triethylamine (40.0 μL, 287.0 μmol) were dissolved in anhydrous DMF (5 mL), and synthesis was conducted according to the procedure described above to obtain a compound 7d as a red purple solid (17.3 mg, 31% yield).

¹H-NMR (400 MHz, CD₃OD): δ 8.22 (d, 2H, J=8.5 Hz), 8.02 (d, 2H, J=13.5 Hz), 7.99-7.92 (m, 4H), 7.64-7.56 (m, 4H), 7.42 (t, 2H, J=7.6 Hz), 6.25 (d, 2H, J=13.9 Hz), 4.39 (t, 4H, J=7.9 Hz), 3.98-3.88 (m, 4H), 3.74-3.65 (m, 4H), 2.99 (t, 4H, J=7.0 Hz), 2.65 (t, 4H, J=6.5 Hz), 2.33-2.24 (m, 7H), 2.01 (s, 12H), 1.92-1.85 (m, 2H)

HRMS (ESI⁻): calcd. for [M-Na⁺] 889.36741, found: 889.37061.

### (5-5) Synthesis of 2-[2-[3-[2-[1,3-dihydro-3,3-dimethyl-1-(3-sulfopropyl)-2H-benz[e]indol-2-ylidene]ethylidene]-2-(4-methylsulfonyl-1-piperazinyl)-1-cyclohexen-1-yl]ethenyl]-3,3-dimethyl-1-(3-sulfopropyl)-3H-benz[e]indolium, Inner Salt, Sodium Salt (Compound 7e: MSP-Cy)

The compound 4 (50 mg, 60.9 μmol), 1-sulfonylpiperazine (29.6 mg, 199.7 μmol) and triethylamine (40.0 μL, 287.0 μmol) were dissolved in anhydrous DMF (5 mL), and synthesis was conducted according to the procedure described above to obtain a compound 7e as a blue solid (11.6 mg, 20% yield).

¹H-NMR (400 MHz, CD₃OD): δ 8.24 (d, 2H, J=8.5 Hz), 8.08 (d, 2H, J=13.9 Hz), 8.00-7.93 (m, 4H), 7.65 (d, 2H, 8.5 Hz), 7.60 (t, 2H, J=8.1 Hz), 7.43 (t, 2H, J=7.6 Hz), 6.29 (d, 2H, J=13.5 Hz), 4.41 (t, 4H, J=7.4 Hz), 3.73 (t, 4H, J=10.0 Hz), 3.58 (t, 4H, J=10.0 Hz), 3.12 (s, 3H), 2.99 (t, 4H, J=7.0 Hz), 2.66 (t, 4H, J=6.3 Hz), 2.34-2.25 (m, 4H), 2.04 (s, 12H), 1.92-1.85 (m, 2H)

HRMS (ESI⁻): calcd. for [M-Na⁺] 925.33440, found: 925.33663.

### (5-6) Synthesis of 2-[2-[3-[2-[1,3-dihydro-3,3-dimethyl-1-(3-sulfopropyl)-2H-benz[e]indol-2-ylidene]ethylidene]-2-(4-dimethyl-1-piperaziniumyl)-1-cyclohexen-1-yl]ethenyl]-3,3-dimethyl-1-(3-sulfopropyl)-3H-benz[e]indolium, Inner Salt, Sodium Salt (Compound 7f: DMP-Cy)

The compound 4 (50 mg, 60.9 μmol), the compound 6 (43.6 mg, 180 μmol) and triethylamine (40.0 μL, 287.0 μmol) were dissolved in anhydrous DMF (5 mL). After stirring the resulting solution at 80° C. for 1.5 hours, additional triethylamine (1 mL, 7.2 mmol) was added. After that, synthesis was conducted according to the procedure described above to obtain a compound 7f as a gray solid (8.4 mg). Since the product degrades during NMR measurements, it was analyzed only by HRMS.

HRMS (ESI⁺): calcd. for [M+Na⁺] 899.38465, found: 899.38750.

### (6) Synthesis of 4-(1-piperazinyl)benzonitrile (Compound 8)

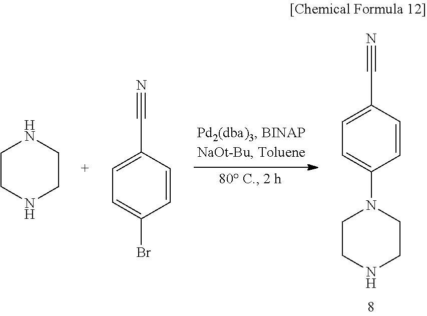 [SMILES: C1CNCCN1 ; N#Cc1ccc(Br)cc1 ; N#Cc1ccc(N2CCNCC2)cc1]

Piperazine (1.15 g, 13.4 mmol), 4-bromobenzonitrile (0.8 g, 4.4 mmol) and NaOt-Bu (0.6 g, 6.2 mmol) were dissolved in anhydrous toluene (20 mL), and (dibenzylideneacetone) dipalladium (9.15 mg, 0.01 mmol) and BINAP (18.7 mg, 0.03 mmol) were added, and after argon substitution, the resulting mixture was stirred at 80° C. for 2 hours. The solution after the reaction was allowed to stand until reaching room temperature, and filtered under reduced pressure using Et₂O. The solvent of the obtained filtrate was removed under reduced pressure, and the obtained product was purified by HPLC (ODS silica column, eluent A: H₂O/0.1% trifluoroacetic acid, eluent B: MeCN/1% H₂O). Then, the solvent was removed under reduced pressure to prepare a solution in which a compound 8 as the desired product was dissolved in water. This solution was purified by freeze drying to obtain a solid of the target compound 8 (180 mg, 22% yield).

¹H-NMR (400 MHz, CD₃OD): δ7.58 (d, 2H, J=9.0 Hz), 7.09 (d, 2H, J=9.0 Hz), 3.57 (t, 4H, J=5.4 Hz), 3.34-3.28 (m, 5H) LRMS (ESI⁺): calcd. for [M+H⁺] 188.13, found: 188.24.

### (7) Synthesis of 4-(4-aminophenyl)piperazine (Compound 9)

4-(4-Nitrophenyl)piperazine (170 mg, 0.82 mmol) was dissolved in anhydrous THF (8 mL), and after adding Pd/C (50 mg, 0.12 mmol), the flask was filled with hydrogen gas. and the resulting mixture was stirred at room temperature for 6 hours. After filtering the solution after the reaction under reduced pressure using THF, the filtrate was subjected to HPLC (ODS silica column eluent A: H₂O/0.1% trifluoroacetic acid, eluent B: MeCN/1% H₂O, gradient conditions: eluent A:eluent B=80:10 to 100:0 (liquid volume ratio)) to confirm the progress of the reaction. Since almost no by-products were observed, a compound 9 obtained as the desired product by removing the solvent under reduced pressure was used in the next reaction.

LRMS (ESI⁺): calcd. for [M+H⁺] 178.14, found: 178.29.

### (8) Synthesis of ICG Derivative (Aryl Piperazine Cyanine)

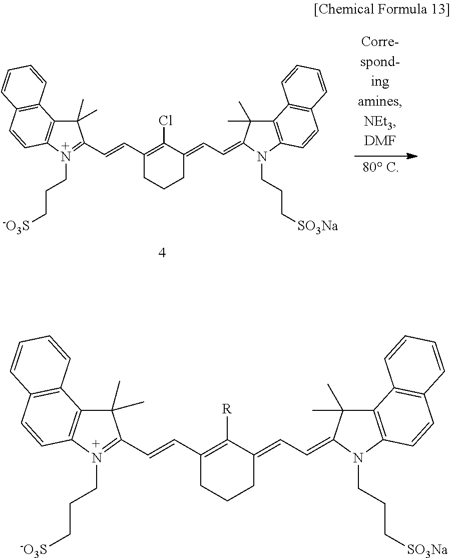

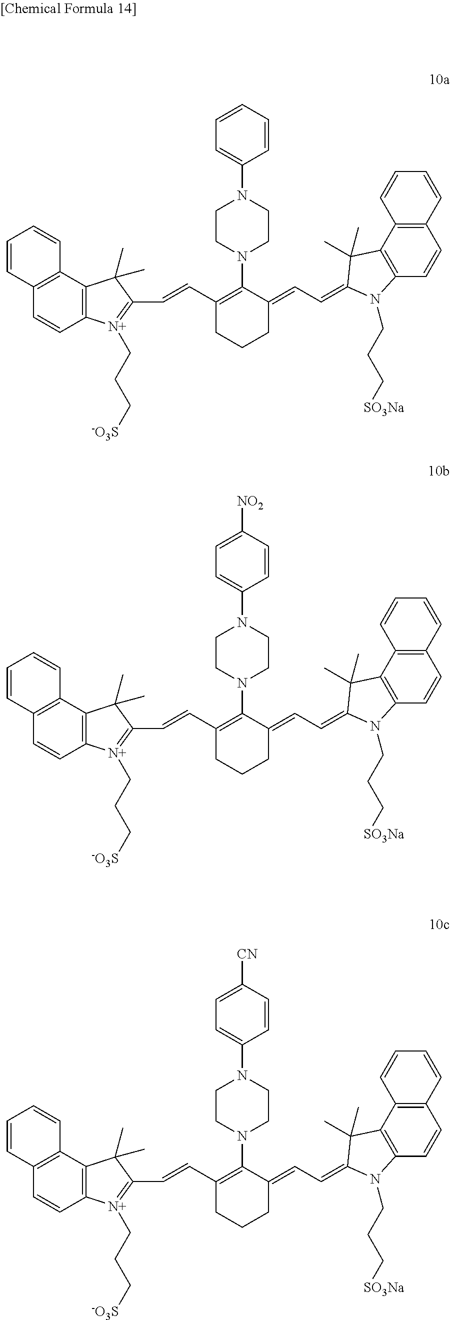

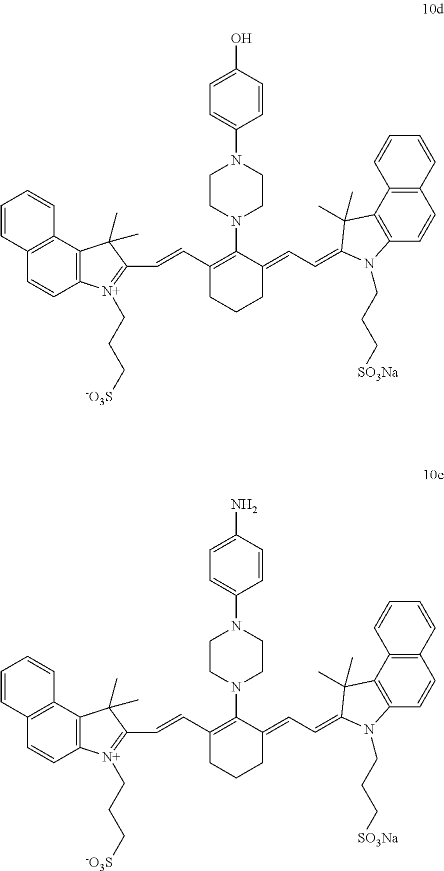

The compound 4 (1 eq.) and the corresponding amine (3 eq.) were dissolved in anhydrous DMF (0.1 to 0.2 mL/μmol), and after adding triethylamine (5 to 7 eq.) thereto and performing argon substitution, the resulting mixture was stirred at 80° C. After confirming the progress of the reaction by HPLC (ODS silica gel, eluent A: 20 mM triethylamine, eluent B: MeCN/1% H₂O, gradient condition: eluent A:eluent B=60:40 to 0:100), the solvent was removed under reduced pressure, and the product was purified by preparative HPLC under the same conditions. The obtained solution was passed through a cation exchange resin, and the solvent was removed under reduced pressure to prepare a solution in which an ICG derivative as the desired product was dissolved in water. This solution was freeze dried to obtain a solid of the target ICG derivative.

The raw materials and yields of each compound are shown below.

### (8-1) Synthesis of 2-[2-[3-[2-[1,3-dihydro-3,3-dimethyl-1-(3-sulfopropyl)-2H-benz[e]indol-2-ylidene]ethylidene]-2-[1-(4-phenyl)-piperazinyl]-1-cyclohexen-1-yl]ethenyl]-3,3-dimethyl-1-(3-sulfopropyl)-3H-benz[e]indolium, Inner Salt, Sodium Salt (Compound 10a: PhP-Cy)

The compound 4 (50 mg, 60.9 μmol), 1-phenylpiperazine (28.35 μL, 180.0 μmol) and triethylamine (40.0 μL, 287.0 μmol) were dissolved in anhydrous DMF (10 mL), and synthesis was conducted according to the procedure described above to obtain a solid of the target compound 10a (8 mg, 14% yield).

¹H-NMR (400 MHz, CD₃OD): δ8.14 (d, 2H, J=9.0 Hz), 8.05 (d, 2H, J=13.9 Hz), 7.97-7.90 (m, 4H), 7.60 (d, 2H, J=9.0 Hz), 7.55 (t, 2H, J=7.9 Hz), 7.43-7.34 (m, 4H), 7.15 (d, 2H, J=8.1 Hz), 6.98 (t, 1H, J=7.2 Hz), 6.22 (d, 2H, J=13.9 Hz), 4.37 (t, 4H, J=7.4 Hz), 3.92-3.86 (m, 4H), 3.64-3.58 (m, 4H), 2.99 (t, 4H, J=7.0 Hz), 2.65 (t, 4H, J=6.5 Hz), 2.32-2.24 (m, 4H), 1.97 (s, 12H), 1.93-1.86 (m, 2H)

HRMS (ESI⁻): calcd. for [M-Na⁺] 923.38815, found: 923.38910.

### (8-2) Synthesis of 2-[2-[3-[2-[1,3-dihydro-3,3-dimethyl-1-(3-sulfopropyl)-2H-benz[e]indol-2-ylidene]ethylidene]-2- [1-(4-nitrophenyl)-piperazinyl]-1-cyclohexen-1-yl]ethenyl]-3,3-dimethyl-1-(3-sulfopropyl)-3H-benz[e]indolium, Inner Salt, Sodium Salt (Compound 10b: Nitro-PhP-Cy)

The compound 4 (50 mg, 60.9 μmol), 1-(4-nitrophenyl)piperazine (37.3 mg, 180.0 μmol) and triethylamine (40.0 μL, 287.0 μmol) were dissolved in anhydrous DMF (7 mL), and synthesis was conducted according to the procedure described above to obtain a solid of the target compound 10b (16.2 mg, 16% yield).

¹H-NMR (400 MHz, CD₃OD): δ8.31 (d, 2H, J=9.0 Hz), 8.15 (d, 2H, J=13.5 Hz), 8.02 (d, 2H, J=8.5 Hz), 7.98-7.91 (m, 4H), 7.63 (d, 2H, J=8.5 Hz), 7.56 (t, 2H, J=7.4 Hz), 7.41 (t, 2H, J=6.7 Hz), 7.23 (d, 2H, J=9.0 Hz), 6.28 (d, 2H, J=13.9 Hz), 4.40 (t, 4H, J=7.4 Hz), 4.03-3.97 (m, 4H), 3.77-3.71 (m, 4H), 2.98 (t, 4H, J=6.7 Hz), 2.67 (t, 4H, J=5.2 Hz), 2.32-2.24 (m, 4H), 1.94-1.87 (m, 14H)

HRMS (ESI⁻): calcd. for [M-Na⁺] 968.37323, found: 968.37354.

### (8-3) Synthesis of 2-[2-[3-[2-[1,3-dihydro-3,3-dimethyl-1-(3-sulfopropyl)-2H-benz[e]indol-2-ylidene]ethylidene]-2-[1-(4-cyanophenyl)-piperazinyl]-1-cyclohexen-1-yl]ethenyl]-3,3-dimethyl-1-(3-sulfopropyl)-3H-benz[e]indolium, Inner Salt, Sodium Salt (Compound 10c: Nitrille-PhP-Cy)

The compound 4 (50 mg, 60.9 μmol), the compound 8 (32 mg, 180 μmol) and triethylamine (60.0 μL, 430.5 μmol) were dissolved in anhydrous DMF (7 mL), and synthesis was conducted according to the procedure described above to obtain a solid of the target compound 10c (0.5 mg, 0.8% yield).

¹H-NMR (400 MHz, CD₃OD): δ8.15-8.08 (m, 4H), 7.98-7.92 (m, 4H), 7.71 (d, 2H, J=9.0 Hz), 7.65-7.59 (m, 4H), 7.42 (t, 2H, J=7.2 Hz), 7.24 (d, 2H, J=9.0 Hz), 6.27 (d, 2H, J=13.9 Hz), 4.39 (t, 4H, J=7.9 Hz), 3.90-3.85 (m, 4H), 3.79-3.74 (m, 4H), 2.98 (t, 4H, J=6.7 Hz), 2.67 (t, 4H, J=6.3 Hz), 2.32-2.24 (m, 4H), 1.92 (s, 12H), 1.30-1.27 (m, 2H)

HRMS (ESI⁻): calcd. for [M-Na⁺] 948.38340, found: 948.38612.

### (8-4) Synthesis of 2-[2-[3-[2-[1,3-dihydro-3,3-dimethyl-1-(3-sulfopropyl)-2H-benz[e]indol-2-ylidene]ethylidene]-2-[1-(4-hydroxyphenyl)-piperazinyl]-1-cyclohexen-1-yl]ethenyl]-3,3-dimethyl-1-(3-sulfopropyl)-3H-benz [e] indolium, Inner Salt, Sodium Salt (Compound 10d: Hydroxy-PhP-Cy)

The compound 4 (50 mg, 60.9 μmol), 1-(4-hydroxyphenyl)piperazine (32 mg, 180.0 μmol) and triethylamine (60.0 μL, 430.5 μmol) were dissolved in anhydrous DMF (7 mL), and synthesis was conducted according to the procedure described above to obtain a solid of the target compound 10d (8.3 mg, 14% yield).

¹H-NMR (400 MHz, CD₃OD): δ8.16 (d, 2H, J=9.0 Hz), 8.02 (d, 2H, J=13.5 Hz), 7.97-7.91 (m, 4H), 7.62-7.54 (m, 4H), 7.40 (t, 2H, J=8.1 Hz), 7.04 (d, 2H, J=9.0 Hz), 6.84 (d, 2H, J=9.0 Hz), 6.21 (d, 2H, J=13.9 Hz), 4.36 (t, 4H, J=10.0 Hz), 3.93-3.88 (m, 4H), 3.47-3.42 (m, 4H), 2.99 (t, 4H, J=7.0 Hz), 2.65 (t, 4H, J=6.5 Hz), 2.31-2.25 (m, 4H), 1.99 (s, 12H), 1.91-1.86 (m, 3H)

HRMS (ESI⁻): calcd. for [M-Na⁺] 939.38306, found: 939.38540.

### (10-5) Synthesis of 2-[2-[3-[2-[1,3-dihydro-3,3-dimethyl-1-(3-sulfopropyl)-2H-benz[e]indol-2-ylidene]ethylidene]-2-[1-(4-aminophenyl)-piperazinyl]-1-cyclohexen-1-yl]ethenyl]-3,3-dimethyl-1-(3-sulfopropyl)-3H-benz[e]indolium, Inner Salt, Sodium Salt (Compound 10e: Amino-PhP-Cy)

The compound 4 (50 mg, 60.9 μmol), 1-(4-aminophenyl)piperazine (31.9 mg, 180.0 μmol) and triethylamine (60.0 μL, 430.5 μmol) were dissolved in anhydrous DMF (7 mL), and synthesis was conducted according to the procedure described above to obtain a solid of the target compound 10e (25 mg, 43% yield).

¹H-NMR (400 MHz, CD₃OD): δ8.15 (d, 2H, J=8.5 Hz), 8.00 (d, 2H, J=13.5 Hz), 7.97-7.90 (m, 4H), 7.62-7.53 (m, 4H), 7.40 (t, 2H, J=7.2 Hz), 7.01 (d, 2H, J=9.0 Hz), 6.83 (d, 2H, J=8.5 Hz), 6.20 (d, 2H, J=13.5 Hz), 4.36 (t, 4H, J=7.6 Hz), 3.92 (s, 4H), 3.41 (s, 4H), 2.99 (t, 4H, J=7.0 Hz), 2.64 (t, 4H, J=6.5 Hz), 2.32-2.23 (m, 4H), 1.99 (s, 12H), 1.92-1.85 (m, 2H)

HRMS (ESI⁻): calcd. for [M-Na⁺] 938.39905, found: 939.40123.

### (9) Synthesis of 4-(1-piperazinylmethyl)-benzeneacetic acid (Compound 11)

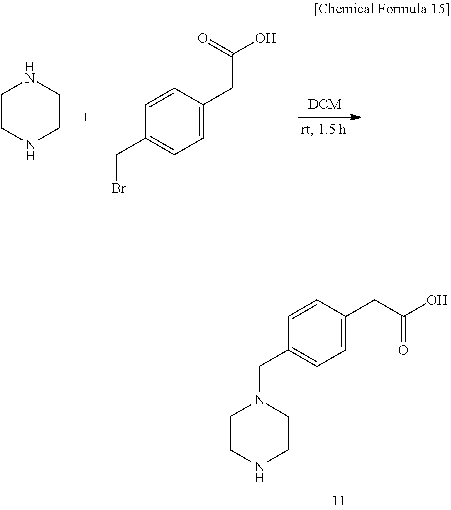 [SMILES: C1CNCCN1 ; O=C(O)Cc1ccc(CBr)cc1 ; O=C(O)Cc1ccc(CN2CCNCC2)cc1]

4-(Bromomethyl)-benzeneacetic acid (1.6 g, 7 mmol) and piperazine (1.8 g, 21 mmol) were dissolved in DCM (15 mL), and the resulting solution was stirred at room temperature for 1.5 hours. After confirming the progress of the reaction by HPLC (ODS silica column, H₂O containing 0.1% TFA/MeCN containing 1% H₂O), the solvent was removed under reduced pressure. The obtained product was subjected to suction filtration using MeCN, and after adding H₂O/0.1% TFA (5 mL) to the filtrate, the resulting mixture was concentrated under reduced pressure to a total volume of about 8 mL. The obtained concentrate was purified by preparative HPLC under the same conditions, and the solvent was removed under reduced pressure to obtain a compound 11 as a white solid (170.6 mg, 10% yield). Since the compound 11 as the desired product was difficult to separate from by-products, it was analyzed only by LRMS and used in the next reaction.

LRMS (ESI⁻): calcd. for [M+H⁺] 235.15, found: 235.12.

### <Measurement of Optical Absorption Spectrum>

In order to investigate the optical absorption properties of the synthesized ICG derivatives, absorption spectra were measured and their pH dependence was examined using an ultraviolet visible spectrophotometer at a concentration of 1 μM either in dimethyl sulfoxide (DMSO) or in phosphate buffers of various pH with DMSO as a cosolvent (30% by volume of DMSO and 70% by volume of phosphate buffer).

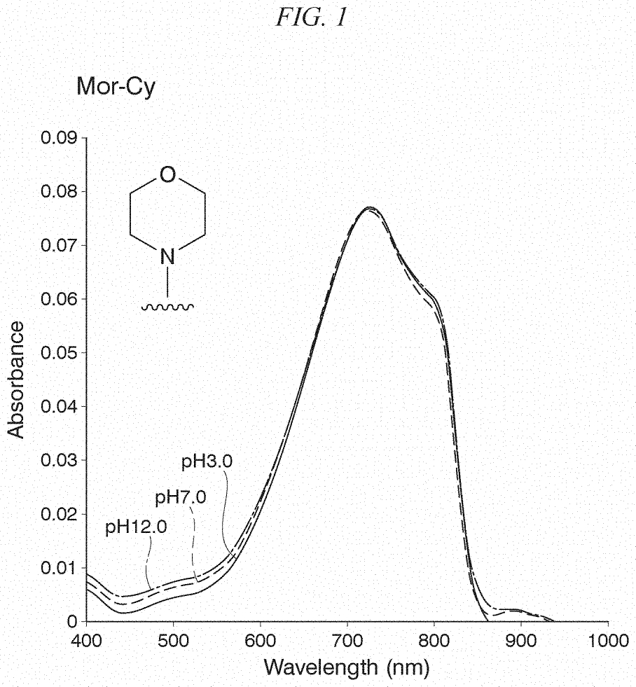

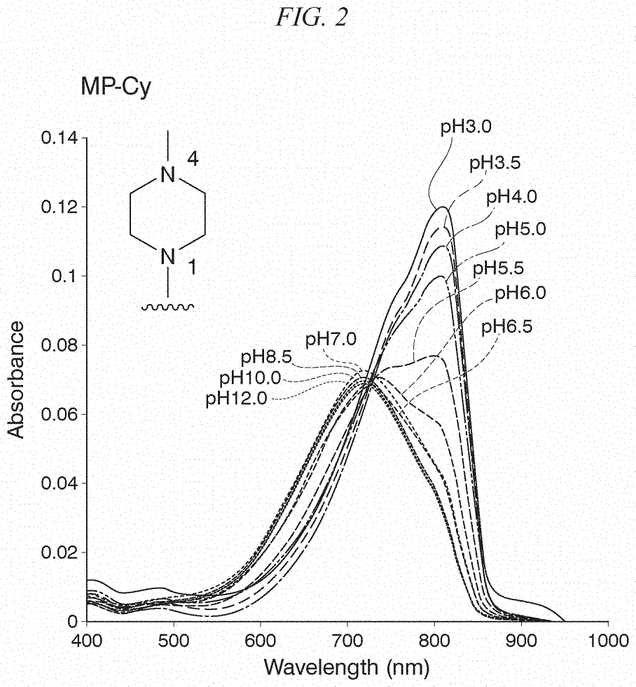

As a result, the pH responsiveness differed depending on the structure of the nitrogen-containing group introduced into the cyclohexene ring, and while the pH had little effect on the optical absorption properties of some ICG derivatives, the optical absorption properties of some ICG derivatives changed greatly in an acidic environment, exhibiting an absorption maximum at around 700 nm in a neutral environment, but the absorption at around 800 nm increased in an acidic environment. Absorption spectra of the ICG derivative Mor-Cy (compound 7b) and the ICG derivative MP-Cy (compound 70 are shown in FIGS. 1 and 2. The ICG derivative Mor-Cy in which the ring bonded to the cyclohexene ring was a morpholine ring showed no change in absorption properties due to the pH (FIG. 1). On the other hand, in the ICG derivative MP-Cy in which the ring bonded to the cyclohexene ring is a piperazine ring, the maximum absorption wavelength shifted to the longer wavelength side as the pH became more acidic.

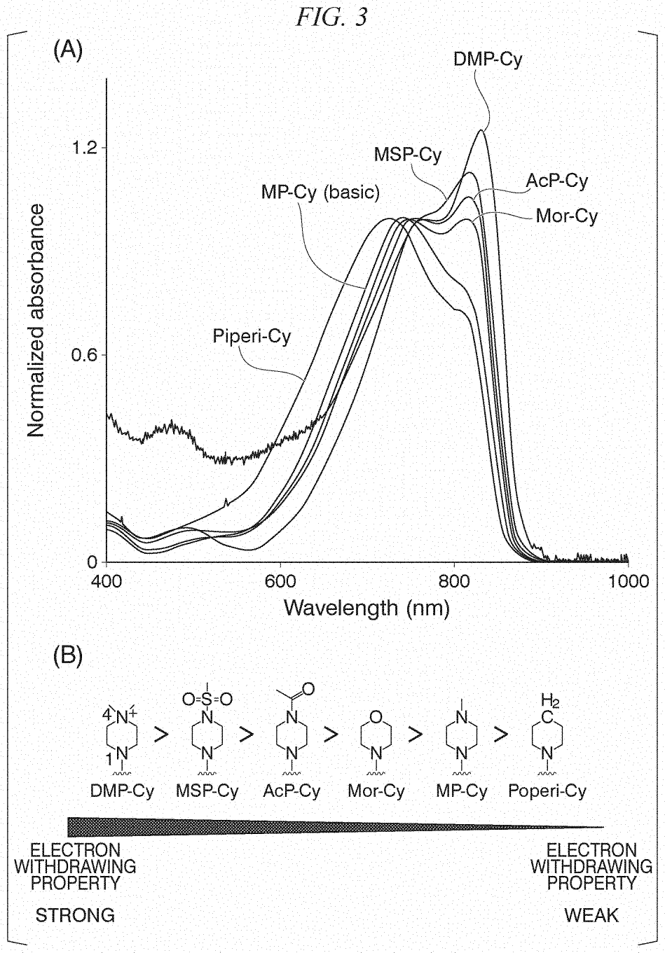

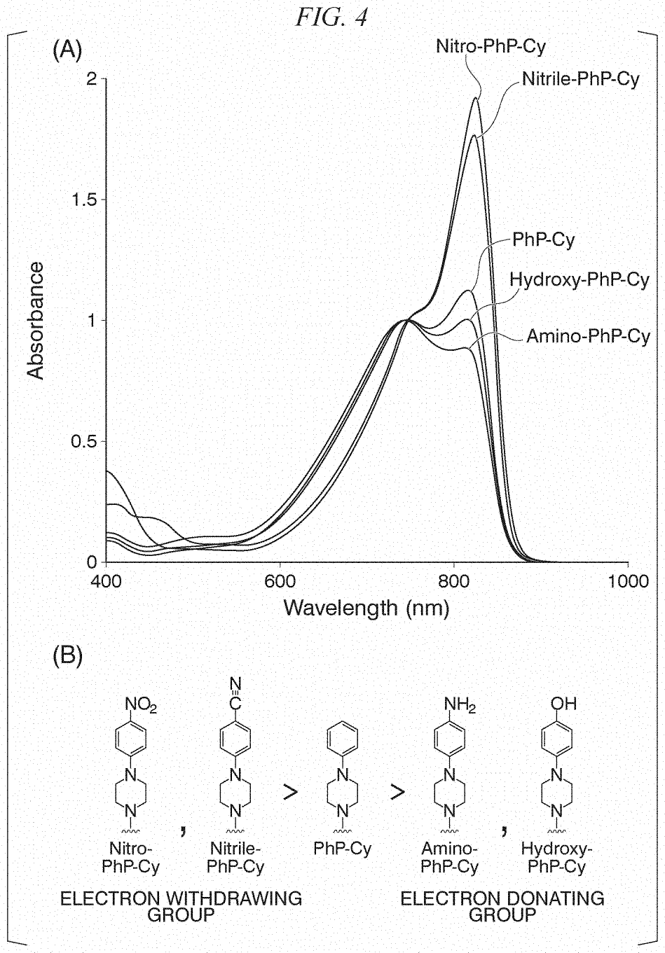

For each ICG derivative, an absorption spectrum consisted of relative absorbance was obtained by setting the absorbance of the absorption peak near 700 nm in DMSO (absorbance at the maximum absorption wavelength) to 1. FIG. 3(A) shows the absorption spectra consisted of the relative absorbance of each ICG derivative (aminocyanine) in DMSO, and FIG. 4(A) shows the absorption spectra consisted of the relative absorbance of each ICG derivative (aryl piperazine cyanine) in DMSO.

As shown in FIG. 3(A), similar to Mor-Cy, Piperi-Cy and MP-Cy had absorption maxima near 700 nm, whereas the absorption maxima of AcP-Cy, MSP-Cy, and DMP-Cy shifted to around 800 nm. As shown in FIG. 3(B), the electron withdrawing properties of the nitrogen-containing group bonded to the cyclohexene ring were such that AcP-Cy<MSP-Cy<DMP-Cy, showing that the maximum absorption wavelength shifted to the longer wavelength side as the electron withdrawing properties of the nitrogen-containing group of the ICG derivative increased.

As shown in FIGS. 4(A) and 4(B), also among the ICG derivatives (aryl piperazine cyanine), in Nitrille-PhP-Cy and Nitro-PhP-Cy where the electron withdrawing properties of the nitrogen-containing group bonded to the cyclohexene ring were strong, the maximum absorption wavelength was shifted to the longer wavelength side.

### <Measurement of L (C—N) Value and Negative Charge Amount Value of Nitrogen Atom Bonded to Cyclohexene Ring>

The negative charge amount value of the nitrogen atom that binds to L(C—N) and the cyclohexene ring in the ICG derivative was measured as follows.

First, the CONFLEX 8 program was used in combination with the B3LYP/3-21G* calculation by the Gaussian 16 program to perform a conformational search for each ICG derivative. Among the obtained conformations, structures with the lowest energy within 3 kcal/mol were subjected to structural optimization calculations by LC-ωRBE/cc-pVDZ calculations using the Gaussian 16 program, as a result of which the most stable conformational structure was specified.

L(C—N) was measured based on the most stable conformational structure specified.

Furthermore, for this specified most stable conformational structure, the amount of charge (electron density) of the nitrogen atom of the piperazine ring bonded to the cyclohexene ring was calculated using natural population analysis.

Tables 1 and 2 show the calculation results of L (C—N) and the charge amount of the nitrogen atom bonded to the cyclohexene ring of each ICG derivative. As a result, a tendency was observed that the longer the L(C—N) of the ICG derivative, and the smaller the amount of charge of the nitrogen atom bonded to the cyclohexene ring (the larger the amount of negative charge), the more the maximum absorption wavelength shifted to the longer wavelength side.

TABLE 1

| | | Amount of charge of |
|---|---|---|
| | L (C—N) | nitrogen atom bonded |
| ICG derivative | [Å] | to cyclohexene ring |
| Piperi-Cy | 1.3366 | −0.43952 |
| MP-Cy | 1.3370 | −0.44158 |
| Mor-Cy | 1.3626 | −0.49497 |
| AcP-Cy | 1.3764 | −0.52485 |
| MSP-Cy | 1.4180 | −0.60233 |
| DMP-Cy | 1.4238 | −0.70135 |

TABLE 2

| | | Amount of charge of |
|---|---|---|
| | L (C—N) | nitrogen atom bonded |
| ICG derivative | [Å] | to cyclohexene ring |
| Hydroxy-PhP-Cy | 1.3735 | −0.52245 |
| Amino-PhP-Cy | 1.3735 | −0.52250 |
| PhP-Cy | 1.3748 | −0.52568 |
| Nitrille-PhP-Cy | 1.3789 | −0.5341 |
| Nitro-PhP-Cy | 1.4100 | −0.59298 |

### Example 2

An ICG derivative bound to an antibody was produced. Trastuzumab or panitumumab was used as an antibody.

### (1) Synthesis of 2-[2-[3-[2-[1,3-dihydro-3,3-dimethyl-1-(3-sulfopropyl)-2H-benz[e]indol-2-ylidene]ethylidene]-2-[1-(4-carboxymethylbenzyl)-piperazinyl]-1-cyclohexen-1-yl]ethenyl]-3,3-dimethyl-1-(3-sulfopropyl)-3H-benz[e]indolium, Inner Salt, Sodium Salt (Compound 12: PBA-Cy)

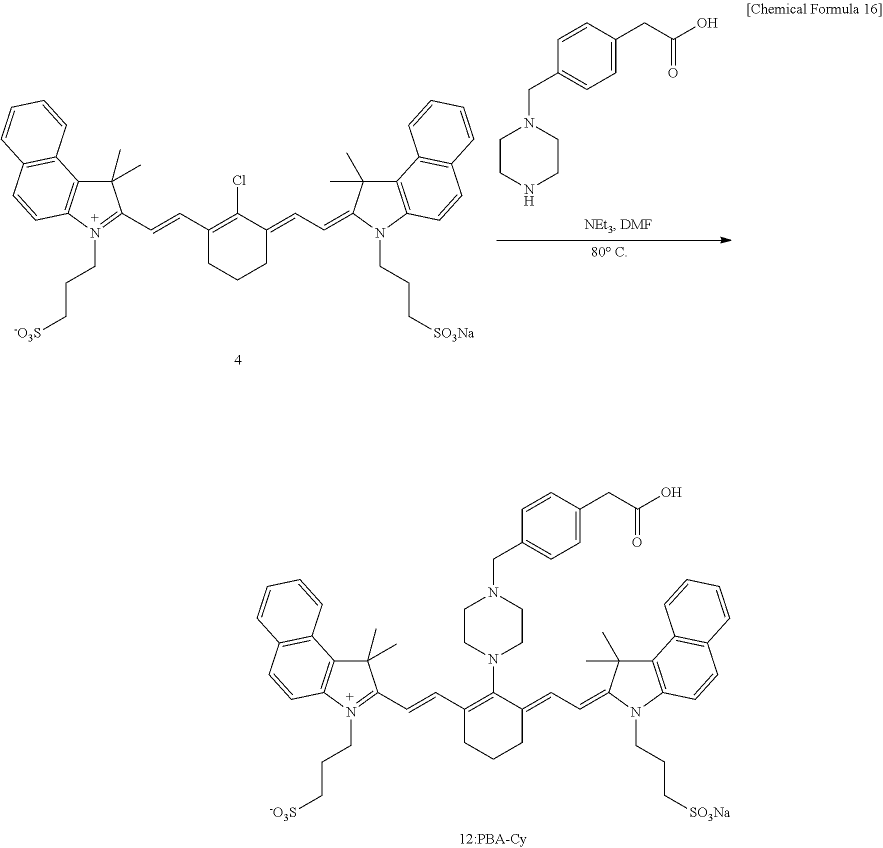

The compound 4 (54.2 mg, 66.0 μmol) and the compound 11 (49.2 mg, 210.0 μmol) were dissolved in anhydrous DMF (3 mL), and after adding triethylamine (50 μL, 359 μmol) thereto and performing argon substitution, the resulting mixture was stirred at 80° C. for 1.5 hours. After confirming the progress of the reaction by HPLC (ODS silica gel, 20 mM TEA aqueous solution/1% H₂O-containing MeCN), the solvent was removed under reduced pressure, and the obtained product was purified by preparative HPLC under the same conditions. The obtained solution was passed through a cation exchange resin, and the solvent was removed under reduced pressure to prepare a solution in which a compound 12 as the desired product was dissolved in water. This solution was freeze dried to obtain the compound 12 as a red solid (12.5 mg, 19% yield).

¹H-NMR (400 MHz, CD₃OD): δ8.21 (d, 2H, J=8.5 Hz), 7.97-7.92 (m, 4H), 7.88 (d, 2H, J=13.5 Hz), 7.64 (t, 2H, J=7.2 Hz), 7.59 (d, 2H, J=9.0 Hz), 7.44-7.38 (m, 6H), 6.15 (d, 2H, J=13.5 Hz), 4.33 (t, 4H, J=7.6 Hz), 3.86-3.80 (m, 6H), 3.50 (s, 2H), 2.99 (t, 4H, J=7.0 Hz), 2.90-2.83 (m, 4H), 2.60 (t, 4H, J=6.5 Hz), 2.30-2.23 (m, 4H), 1.97 (s, 12H), 1.90-1.82 (m, 2H)

HRMS (ESI⁻): calcd. for [M-Na⁺] 995.40928, found: 995.41094.

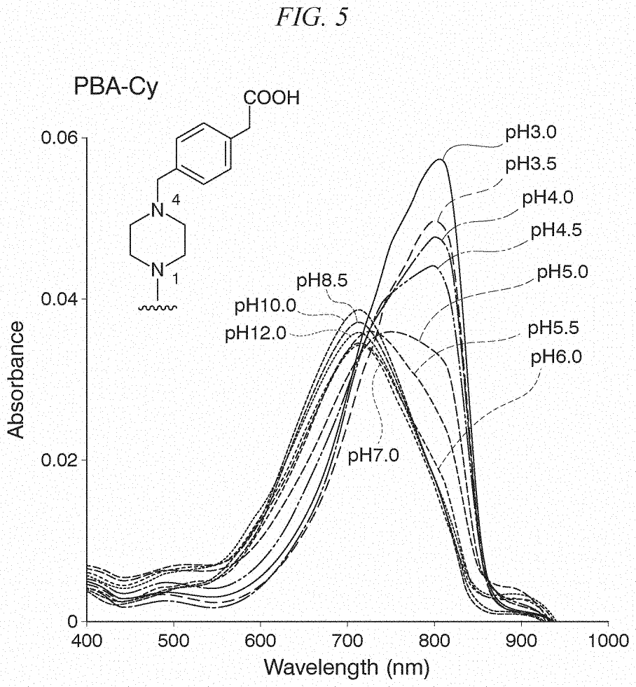

The optical absorption spectra of the IGC derivative PBA-Cy was measured at various pH values in the same manner as in Example 1. The measurement results are shown in FIG. 5. The maximum absorption wavelength of the IGC derivative PBA-Cy shifted to the longer wavelength side as the pH became more acidic.

### (2) Synthesis of PBA-Cy-Antibody Conjugate (Conjugate 14: Tra-PBA-Cy, Conjugate 15: Pan-PBA-Cy)

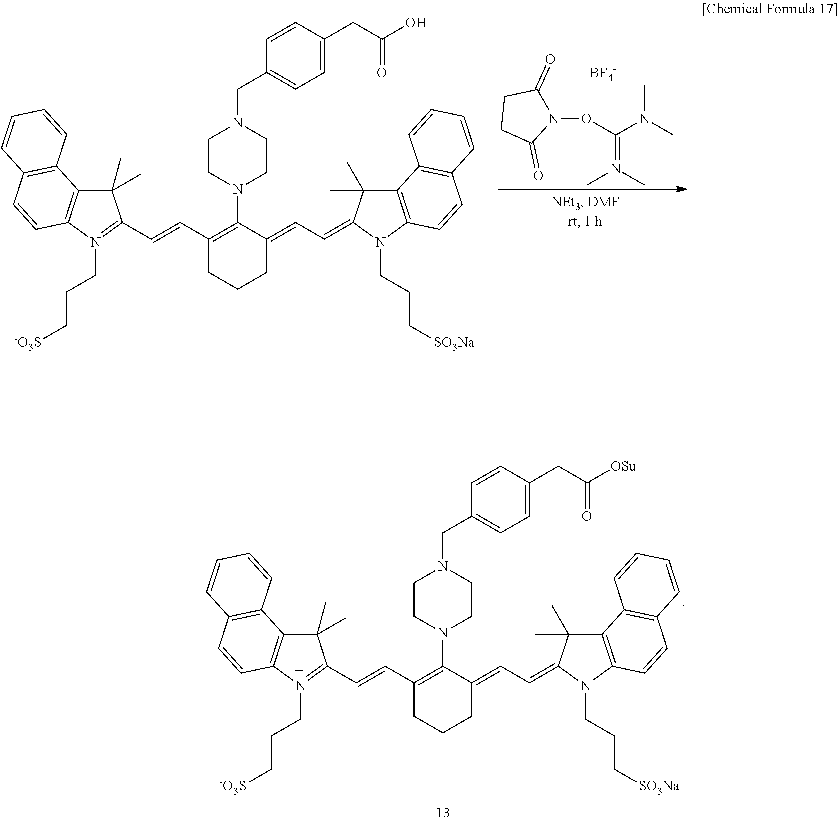

PBA-Cy (2 mg, 2.0 μmol), N,N,N′,N′-tetramethyl-O—(N-succinimidyl)uronium tetrafluoroborate (1.2 mg, 4.0 μmol), and triethylamine (1 μL, 8.9 μmol) were dissolved in DMF (3 mL), and the resulting solution was stirred at room temperature for 1 hour. After confirming the progress of the reaction by HPLC (ODS silica gel, eluent A: 0.1 M triethylamine acetate buffer, eluent B: MeCN containing 1% H₂O), purification was performed by preparative HPLC under the same conditions. After removing the solvent under reduced pressure to remove the triethylamine acetate salt, the solvent was removed again under reduced pressure to obtain a compound 13 as the desired product. After confirming the purity by HPLC, this target product was directly used for the next reaction.

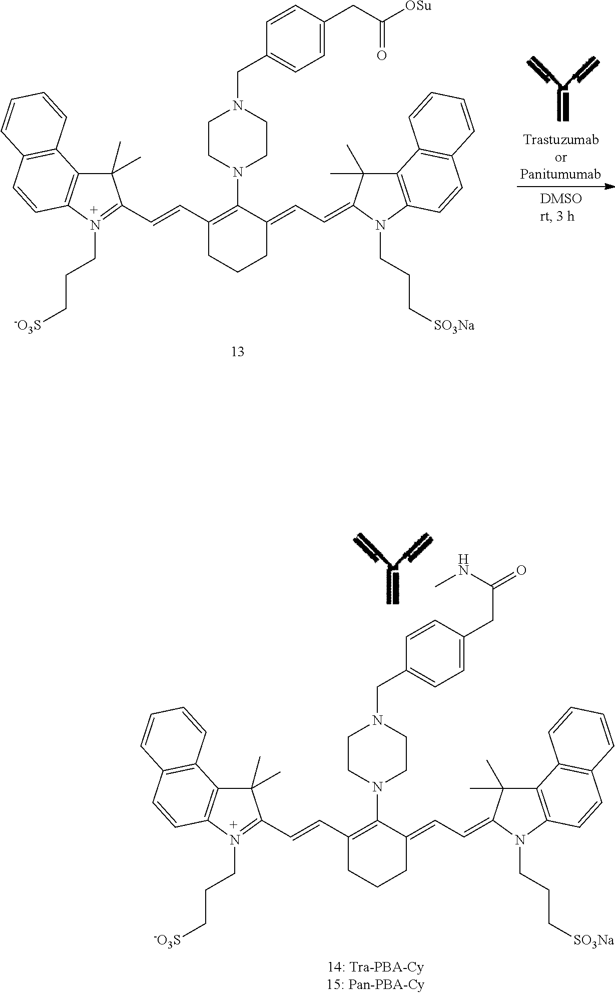

The compound 13 was dissolved in DMSO (10 μL) to prepare a 48.75 mM DMSO solution. This DMSO solution of the compound 13 (0.32 μL, 13.5 nmol) and trastuzumab (400 μg, 2.7 nmol) or panitumumab (400 μg, 2.7 nmol) were added to a 0.1 M Na₂HPO aqueous solution so that the total volume was 300 μL, and the resulting mixture was allowed to stand at room temperature for 3 hours for reaction. The solution after the reaction was purified using Amicon. The protein concentration in the solution after purification was measured using a BCA protein assay kit (manufactured by Thermo Fisher Scientific Inc.). The concentration of PBA-Cy in this solution was calculated from the molar absorption coefficient (32,000 M⁻¹ cm⁻¹) using an ultraviolet-visible spectrophotometer (“UV-1800” manufactured by Shimadzu Corporation). As a result, a conjugate 14 (Tra-PBA-Cy) or conjugate 15 (Pan-PBA-Cy) having a molar ratio of antibody:PBA-Cy=1:2 was obtained.

### <Photoacoustic Imaging in Cells>

The obtained antibody conjugate was introduced into cultured cells and photoacoustic imaging was performed. MDA-MB-231 cells derived from human breast cancer were used as cultured cells.

Cells were seeded in a 3.5 cm dish using 2 mL of culture medium (Leibovitz's L-15 medium containing phenol red) and cultured for 24 hours at 37° C. in a 5% CO₂ environment. Then, after removing the medium and washing the cells with 1 mL of measurement buffer (Tyrode solution containing glucose and HEPES), 2 mL of measurement buffer containing 10 μg/mL of PBA-Cy-antibody conjugate (Tra-PBA-Cy or Pan-PBA-Cy) was added for each 3.5 cm dish. Photoacoustics were measured with a transmission microscope in a state where the 3.5 cm dish was kept at 37° C.

The measurement was performed by irradiating a pulse wave multiple times in a range of 2 μm×2 μm and calculating the average of the observed signals. The signal was measured as an absolute value and corrected for the laser intensity at the time of measurement. First, after irradiating with excitation light of 800 nm and measuring photoacoustic signals, the same cells were irradiated with excitation light of 720 nm and photoacoustic signals were measured. After that, the same cells were further irradiated with excitation light of 800 nm, and photoacoustic signals were measured. In the case of excitation light of 800 nm, a pulse wave with an average laser intensity of 18 μJ was irradiated four times in 20 minutes. In the case of excitation light of 720 nm, a pulse wave with an average laser intensity of 20 μJ was irradiated eight times in 40 minutes. The photoacoustic signals were measured in a range of 50 times (in the horizontal direction)×50 times (in the vertical direction) (100 μm×100 μm) per dish.

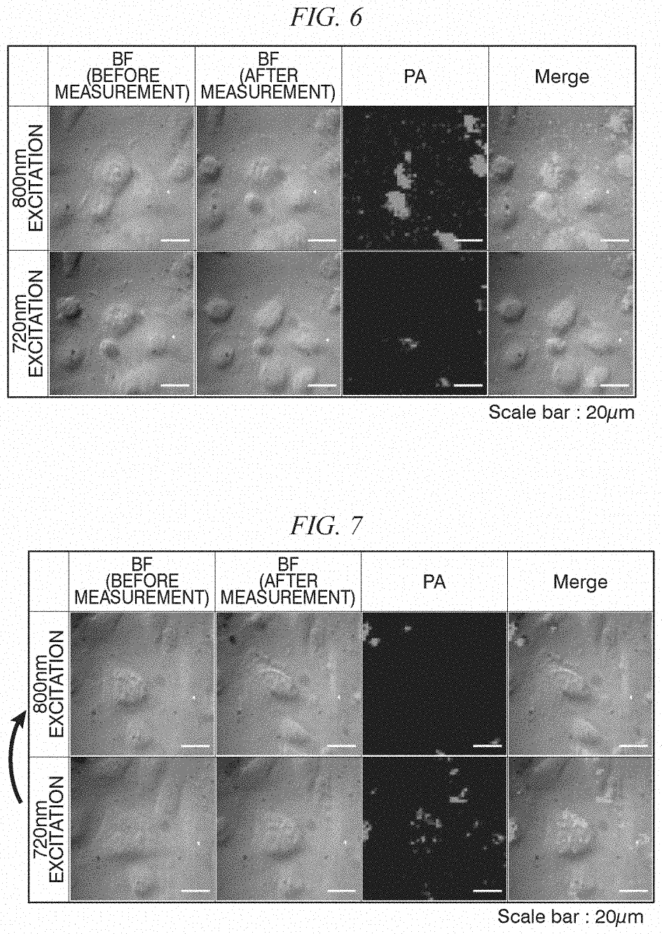

FIG. 6 shows photoacoustic imaging images of cells irradiated for the first time (excitation light of 800 nm) and cells irradiated for the second time (excitation light of 720 nm). In the figure, “BF (before measurement)” shows a bright-field image of cells before irradiation with excitation light, “BF (after measurement)” shows a bright-field image of cells after irradiation with excitation light, and “PA” shows a photoacoustic image of cells at the time of irradiation with excitation light, respectively. As shown in FIG. 6, photoacoustic signals were observed from within the cells when excited at 800 nm. When successively excited at 720 nm, nothing was observed from within the cells. After that, nothing was observed from within the cells even when further excited at 800 nm. The reason why no signal was observed in the third irradiation was considered to be that discoloration of the PBA-Cy-antibody conjugate occurred.

The photoacoustic signals were measured in the same manner except that the first irradiation was performed with an excitation light of 720 nm and then the second irradiation was performed with an excitation light of 800 nm. FIG. 7 shows photoacoustic imaging images of cells irradiated for the first time (excitation light of 720 nm) and cells irradiated for the second time (excitation light of 800 nm). As a result, when excited from 720 nm, only weak signals were observed from within the cells. Even when successively excited at 800 nm, nothing was observed.

### Example 3

An ICG derivative was synthesized and its optical absorption spectrum was measured.

<Synthesis of ICG derivative>

Two types of ICG derivatives having structures represented by the general formula (2) were synthesized by the following synthesis reactions. The synthesized ICG derivative was analyzed by NMR and MS to confirm that an ICG derivative having the desired structure was synthesized.

### (1) Synthesis of 2-[2-[2-[4-(2-carboxyethyl)piperazinyl]-3-[2-[1,3-dihydro-3,3-dimethyl-1-(3-sulfopropyl)-2H-benz[e]indol-2-ylidene]ethylidene]-1-cyclohexen yl]ethenyl]-3,3-dimethyl-1-(3-sulfopropyl)-3H-benz[e]indolium, Inner Salt, Sodium Salt (Compound 16: PPA-Cy)

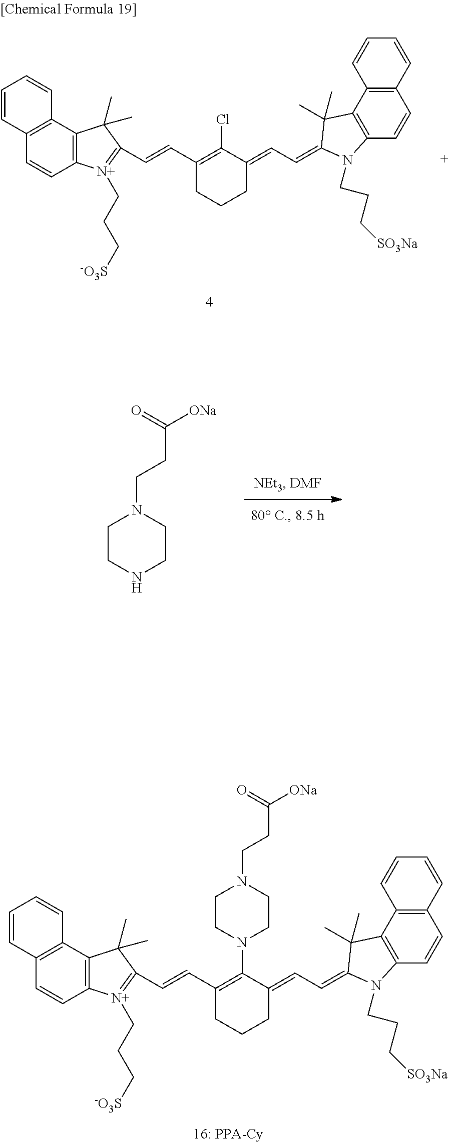

The compound 4 (50.0 mg, 60.9 μmol) and 3-(piperazin-1-yl)propanoate (28.9 mg, 182 μmol) were added to anhydrous DMF (4 mL). Then triethylamine (50.7 μL, 364 μmol) was added thereto, and the resulting mixture was stirred at 80° C. for 8.5 hours. After confirming the progress of the reaction by HPLC under the following conditions, DMF was removed under vacuum, and the precipitated solid was purified by HPLC under the same conditions. After removing MeCN from the collected fractions, they were passed through a Na cation exchange resin. This solution was freeze dried to obtain the compound 16 as a red purple solid (10.5 mg, 18% yield).

HPLC Conditions

Solution A: 20 mM TEA buffer (pH 10 to 11)

Solution B: 99% MeCN/1% H₂O

Liquid flow conditions: 0 to 5 minutes (solution B concentration: 30%, isocratic)→5 to 14 minutes (solution B concentration: 30 to 60%, gradient)

Elution time: 13.7 minutes (λabs=700 nm)

¹H-NMR (400 MHz, CD₃OD): δ 8.22 (d, 2H, J=8.5 Hz), 7.97-7.89 (m, 6H), 7.62-7.56 (m, 4H), 7.40 (t, 2H, J=7.0 Hz), 6.16 (d, 2H, J=13.5 Hz), 4.34 (t, 4H, J=7.6 Hz), 3.84 (t, 4H, J=3.8 Hz), 3.02-2.88 (m, 10H), 2.62 (t, 4H, J=6.5 Hz), 2.54 (t, 4H, J=7.6 Hz), 2.32-2.23 (m, 4H), 2.02 (s, 12H), 1.90-1.84 (m, 2H).

HRMS (ESI⁺): calcd. for C₅₁H₅₈N₄Na₃O₈S₂ [M+Na]⁺987.3384, found: 987.3367.

### (2) Synthesis of 2,3,3-trimethyl-7-sulfo-1H-benz[e]indole, Inner Salt, Potassium Salt (Compound 17)

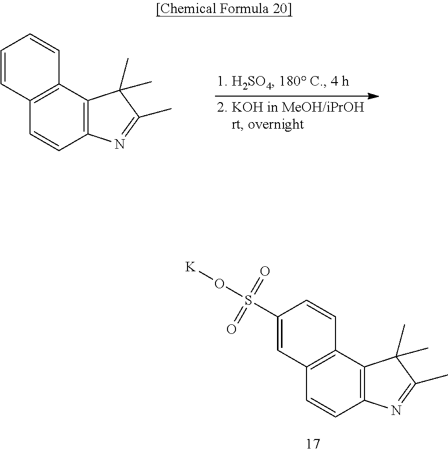

1,1,2-trimethyl-1H-benz[e]indole (4.00 g, 19.1 mmol) was added to concentrated sulfuric acid (16.00 mL, 284.0 mmol), and the resulting mixture was stirred at 180° C. for 4 hours. After the reaction, the reaction solution was cooled to 0° C. and an excess amount of ethyl acetate was added. The produced precipitate was filtered, and the obtained solid was dissolved in a mixed solvent of methanol/isopropanol (MeOH/i-PrOH=3:2, 80 mL) containing potassium hydroxide (4.8 g, 85.8 mmol). After stirring this solution overnight at room temperature, the solvent was removed. The residue was suspended in isopropanol and hexane, and the resulting suspension was filtered to obtain a compound 17 as a blackish brown solid (3.02 g, 48% yield).

¹H-NMR (400 MHz, CD₃OD): δ8.44 (s, 1H), 8.19 (d, 1H, J=8.5 Hz), 8.02-7.96 (m, 2H), 7.74 (d, 1H, J=8.5 Hz), 2.41 (s, 3H), 1.57 (s, 6H)

LRMS (ESI⁻): calcd. for C₁₅H₁₄NO₃S [M−K]⁻ 288, found: 288.

### (3) Synthesis of 2,3,3-trimethyl-1-(3-sulfopropyl)-7-sulfo-1H-benz[e]indolium, Inner Salt, Potassium Salt (Compound 18)

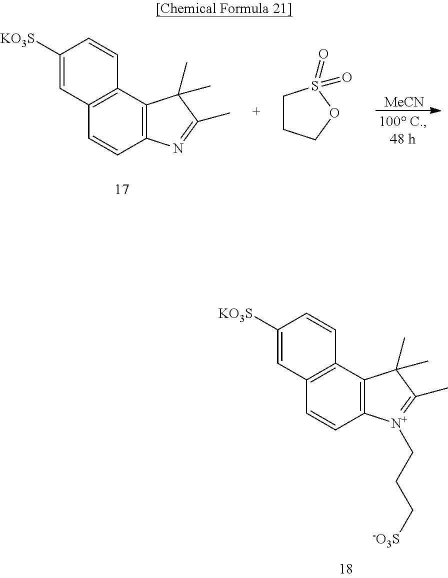

The compound 17 (1.80 g, 5.50 mmol) and propane sultone (2.42 mL, 27.5 mmol) were dissolved in MeCN, and after argon substitution, the resulting solution was stirred while heating under reflux for 48 hours. After confirming the progress of the reaction by HPLC under the following conditions, MeCN was removed under reduced pressure to obtain a compound 18 (2.05 g) as a blackish brown crude product, which was used in the next reaction.

HPLC Conditions

Solution A: 0.1M TEAA buffer

Solution B: 99% MeCN/1% H₂O

Liquid flow conditions: 0 to 2 minutes (solution B concentration: 10%, isocratic)→2 to 28 minutes (solution B concentration: 10 to 50%, gradient)

LRMS (ESI⁻): calcd. for C₁₈H₂₀NO₆S₂ [M−K]⁻ 410, found: 410.

### (4) Synthesis of 2-[2-[2-chloro-3-[2-[1,3-dihydro-3,3-dimethyl-1-(3-sulfopropyl)-7-sulfo-2H-benz[e]indol-2-ylidene]ethylidene]-1-cyclohexen-1-yl]ethenyl]-3,3-dimethyl-1-(3-sulfopropyl)-7-sulfo-3H-benz[e]indolium, Inner Salt, Sodium Salt (Compound 19)

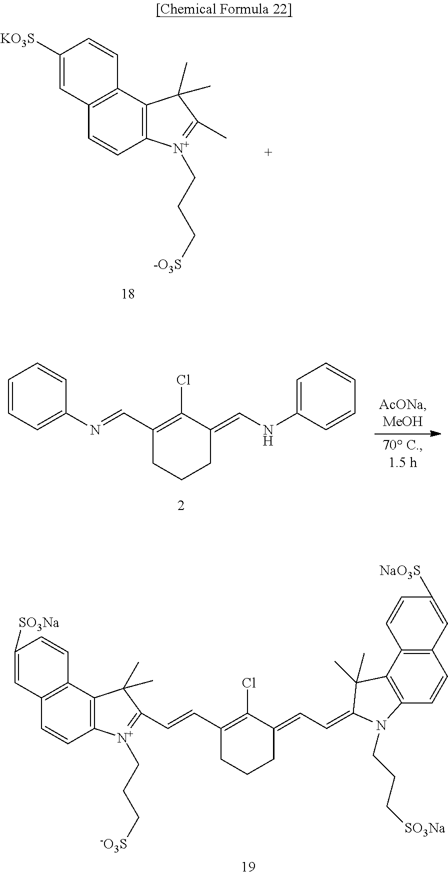

The compound 18 (1.10 g, 2.44 mmol), the compound 2 (394 mg, 1.22 mmol) and sodium acetate (328 mg, 4 mmol) were dissolved in anhydrous methanol (10 mL), and after argon substitution, the resulting solution was stirred at 70° C. for 1.5 hours. After confirming the progress of the reaction by HPLC under the following conditions, methanol was removed under reduced pressure. The precipitated solid was purified by HPLC under the same conditions. After removing MeCN from the collected fraction, a desalination operation was performed. Then the obtained solution was passed through a Na cation exchange resin. This solution was freeze dried to obtain the compound 19 as a green solid (8.69 mg, 0.7% yield).

HPLC Conditions

Solution A: 0.1M TEAA buffer

Solution B: 99% MeCN/1% H₂O

Liquid flow conditions: 0 to 2 minutes (solution B concentration: 10%, isocratic)→2 to 4 minutes (solution B concentration: 10 to 30%, gradient)→4 to 19.5 minutes (solution B concentration: 30 to 50%, gradient)

Elution time: 11.4 minutes

¹H-NMR (400 MHz, CD₃OD): δ8.58 (d, 2H, J=14.4 Hz), 8.44 (s, 2H), 8.35 (d, 2H, J=9.0 Hz), 8.12 (d, 2H, J=9.0 Hz), 8.03 (dd, 2H, J=8.8, 1.6 Hz), 7.79 (d, 2H, J=9.0 Hz), 6.54 (d, 2H, J=13.9 Hz), 4.52 (t, 4H, J=7.6 Hz), 3.03 (t, 4H, J=6.7 Hz), 2.82 (t, 4H, J=5.8 Hz), 2.37-2.27 (m, 4H), 2.09-1.91 (m, 14H).

HRMS (ESI⁻): calcd. for C₄₄H₄₄N₂O₁₂ClNaS₄ [M−2Na]²⁻ 489.0687, found: 489.0702.

### (5) Synthesis of 2-[2-[2-[4-(2-carboxyethyl)piperazinyl]-3-[2-[1,3-dihydro-3,3-dimethyl-1-(3-sulfopropyl)-7-sulfo-2H-benz[e]indol-2-ylidene]ethylidene]cyclohexen-1-yl]ethenyl]-3,3-dimethyl-1-(3-sulfopropyl)-7-sulfo-3H-benz[e]indolium, Inner Salt, Sodium Salt (Compound 20: SS-PPA-Cy)

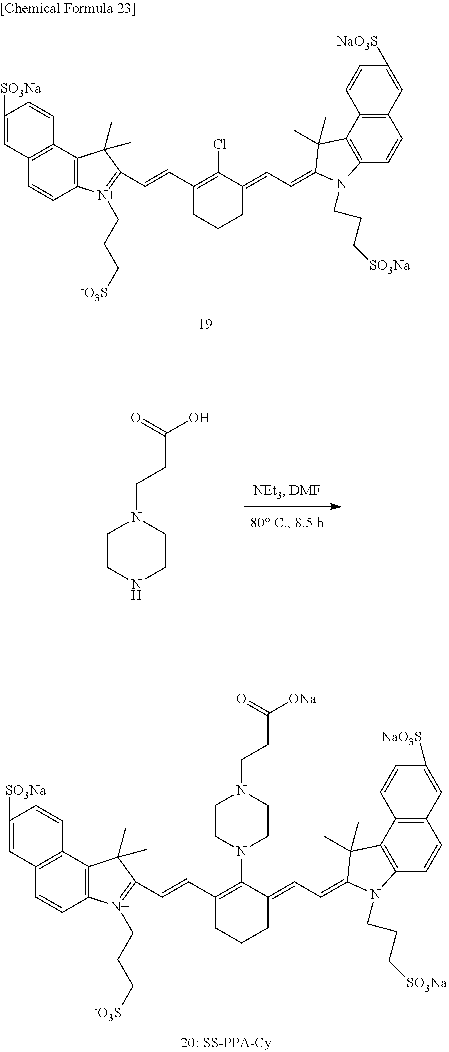

The compound 19 (20.0 mg, 19.5 μmol) and 3-(piperazin-1-yl)propanoate (9.25 mg, 58.5 μmol) were added to anhydrous DMF (4 mL). Then triethylamine (16.3 μL, 117 μmol) was added thereto, and the resulting mixture was stirred at 80° C. for 8.5 hours. After confirming the progress of the reaction by HPLC under the following conditions, DMF was removed under vacuum. The precipitated solid was purified by HPLC under the same conditions. After removing MeCN from the collected fractions, they were passed through a Na cation exchange resin. This solution was freeze dried to obtain the compound 20 as a blue-green solid (13.5 mg, 59% yield).

HPLC Conditions

Solution A: 20 mM TEA buffer (pH 10 to 11)

Solution B: 99% MeCN/1% H₂O

Liquid flow conditions: 0 to 18 minutes (solution B concentration: 10 to 60%, gradient)

Elution time: 11.4 minutes (λabs=700 nm)

¹H-NMR (400 MHz, CD₃OD): δ8.38 (s, 2H), 8.29 (d, 4H, J=9.0 Hz), 8.03 (d, 2H, J=9.0 Hz), 7.98 (dd, 2H, J=9.0, 1.8 Hz), 7.85 (d, 2H, J=13.5 Hz), 7.64 (d, 2H, J=9.0 Hz), 6.16 (d, 2H, J=13.0 Hz), 4.33 (t, 4H, J=7.4 Hz), 3.96-3.88 (m, 4H), 3.03-2.86 (m, 10H), 2.62 (t, 4H, J=6.5 Hz), 2.53 (t, 2H, J=7.6 Hz), 2.32-2.22 (m, 4H), 2.01 (s, 12H), 1.92-1.83 (m, 2H).

HRMS (ESI⁺): calcd. for C₅₁H₅₆N₄Na₅O₁₄S₄ [M+Na]⁺1191.2159, found: 1191.2147.

### <Measurement of Optical Absorption Spectrum>

The optical absorption properties of the synthesized ICG derivatives (PPA-Cy and SS-PPA-Cy) were investigated in the same manner as in Example 1, and their pH dependence was examined.

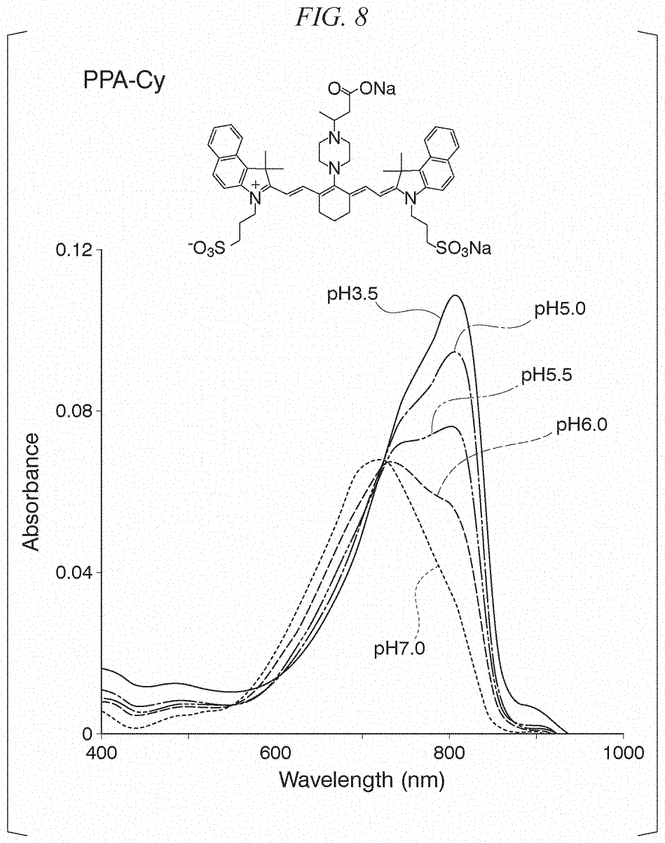

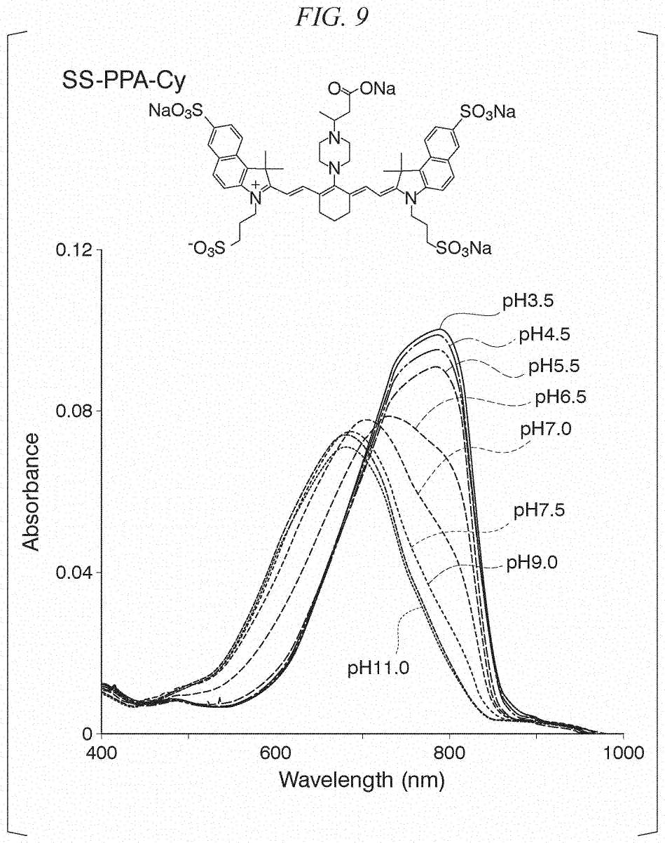

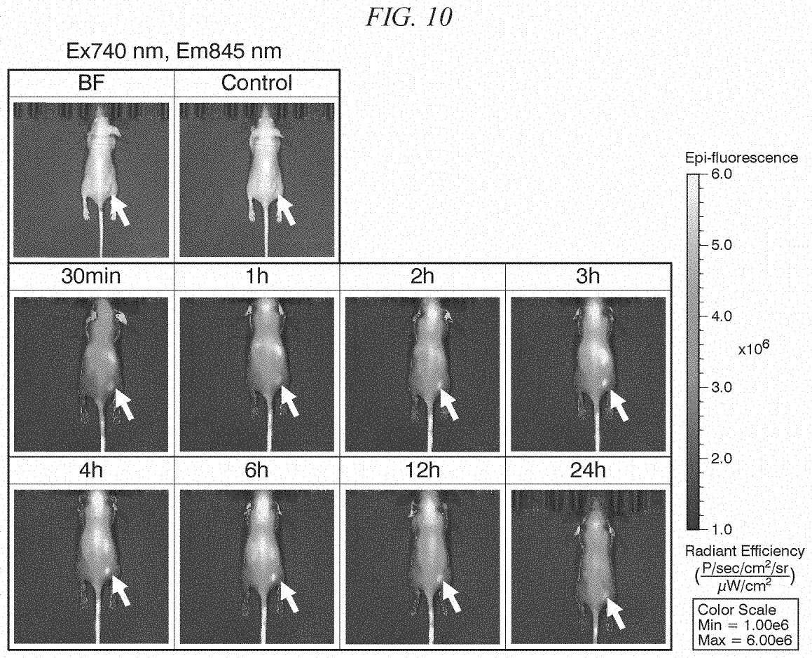

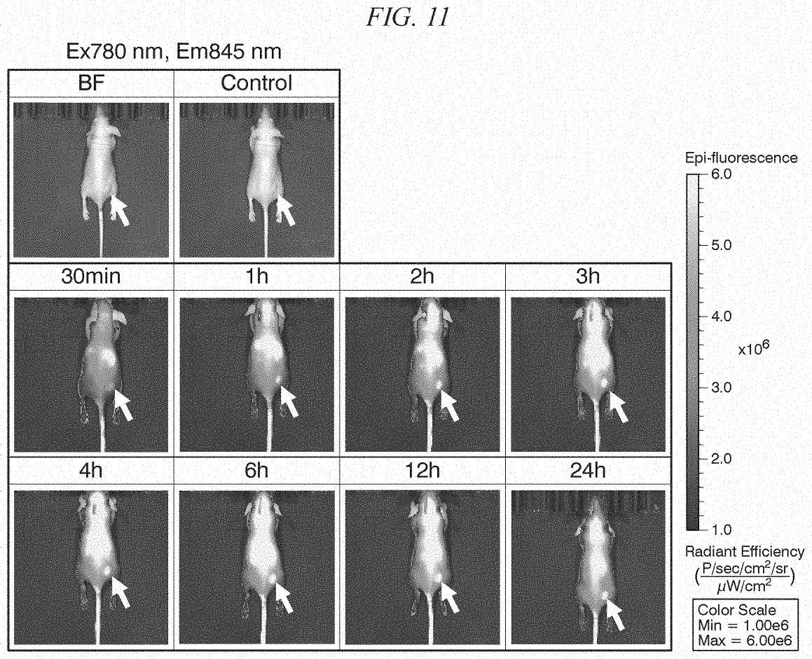

FIGS. 8 and 9 show the absorption spectra of the ICG derivative PPA-Cy (compound 16) and the ICG derivative SS-PPA-Cy (compound 20). As a result, for both ICG derivatives, the maximum absorption wavelength shifted to the longer wavelength side as the pH became more acidic. The pKa of the ICG derivative PPA-Cy was 5.7, which was higher than the pKa (4.9) of the ICG derivative PBA-Cy in which the alkylene group portion in R^{D} was a phenylene group. Furthermore, the pKa of the ICG derivative SS-PPA-Cy in which a sulfo group was introduced to increase hydrophilicity was 6.8, and the pKa was further increased.

### Example 4

An ICG derivative bound to a peptide was produced. An RGD peptide was used as the peptide. As the ICG derivative, the ICG derivative PBA-Cy (compound 12) synthesized in Example 1 and the ICG derivative PPA-Cy (compound 16) synthesized in Example 3 were used.

### (1) Synthesis of ICG Derivative RGD2-PBA-Cy (Compound 21)

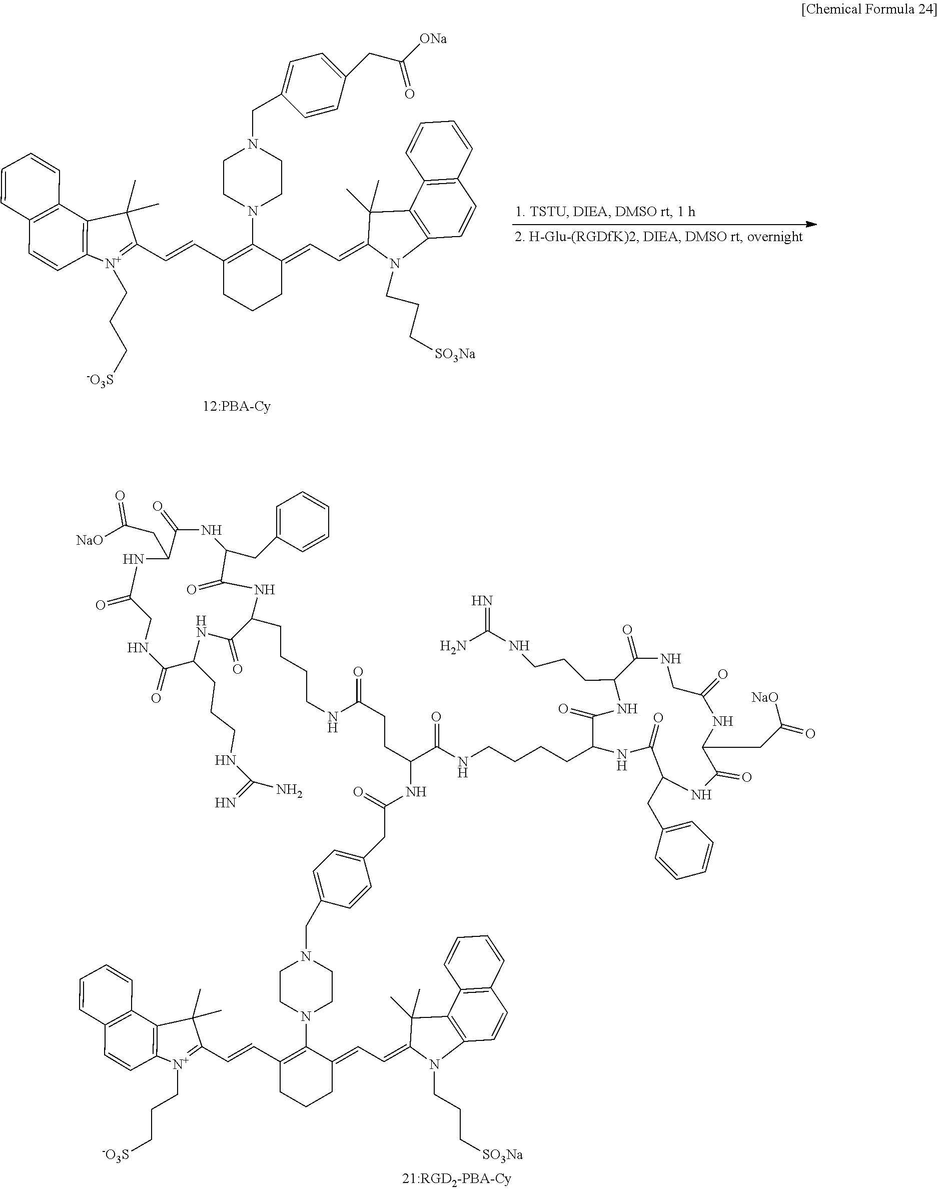

The compound 12 (0.85 mg, 0.76 μmol) and TSTU (CAS No.: 105832-38-0: 0.23 mg, 0.76 μmol) were added to anhydrous DMSO (350 μL). Then DIEA (N,N-diisopropylethylamine: 0.27 μL, 1.52 μmol) was added thereto, and the resulting mixture was stirred at room temperature for 1 hour. After confirming the progress of the reaction by HPLC under the following conditions, H-Glu-[c(RGDfK)]₂ (0.50 mg, 0.38 μmol) was added to the reaction solution and allowed to react overnight at room temperature. After confirming the progress of the reaction by HPLC under the same conditions, the reaction solution was diluted with H₂O/MeCN, and the reactant was fractionated by HPLC. After removing MeCN from the collected fraction, a desalination operation was performed. Then the fraction solution was passed through a Na cation exchange resin. The obtained solution was freeze dried to obtain the compound 21 as a blue solid (1.20 mg, 67% yield). The product was identified by HRMS, and the purity was confirmed by analytical HPLC.

HPLC Conditions

Solution A: 0.1M TEAA buffer

Solution B: 99% MeCN/1% H₂O

Liquid flow conditions: 0 to 29 minutes (solution B concentration: 10 to 80%, gradient)

Elution time: 20 minutes

HRMS (ESI⁺): calcd. for C₁₁₆H₁₄₈N₂₃Na₃O₂₃S₂ [M+2H]²⁺1182.0121, found: 1182.0171.

### (2) Synthesis of ICG Derivative RGD2-PPA-Cy (Compound 22)

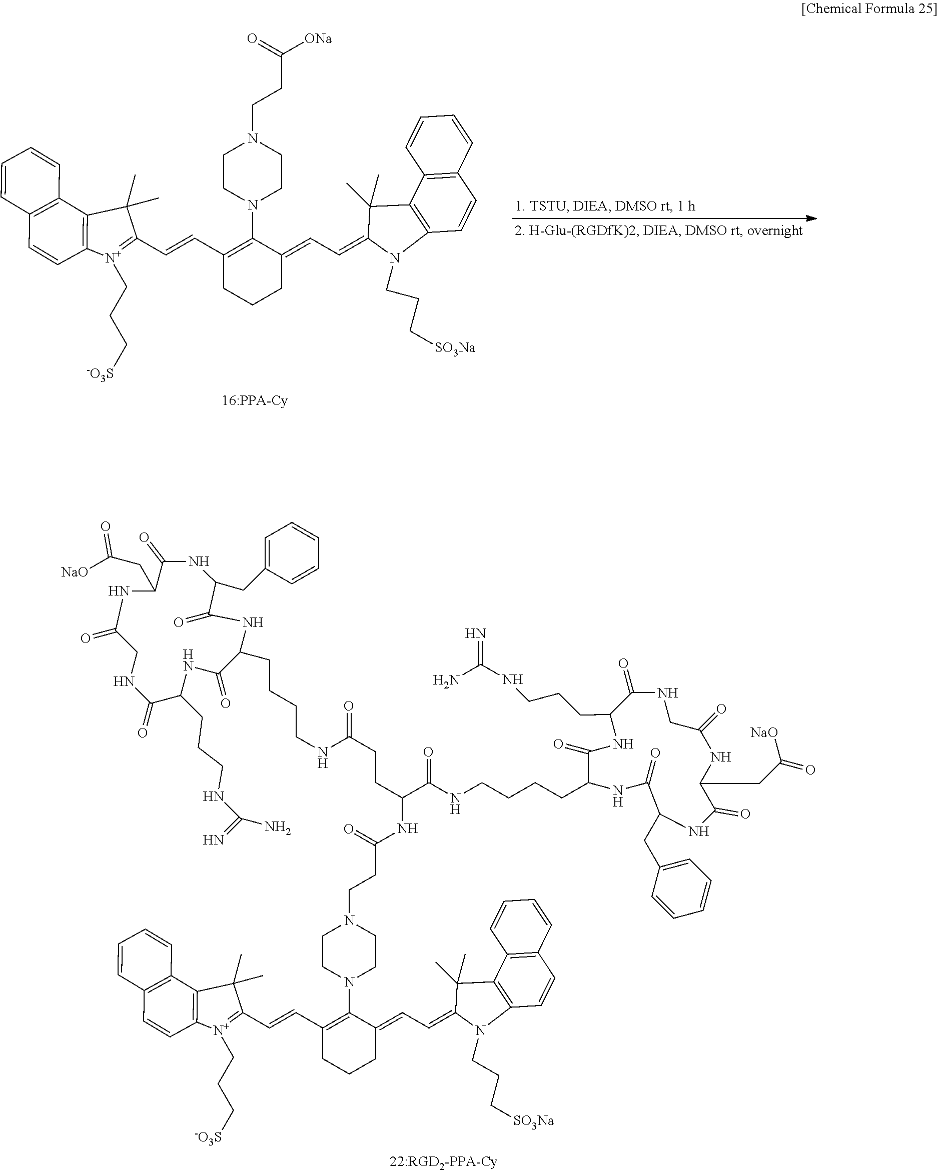

The compound 16 (0.74 mg, 0.76 μmol) and TSTU (0.23 mg, 0.76 μmol) were added to anhydrous DMSO (350 μL). Then DIEA (0.27 μL, 1.52 μmol) was added thereto, and the resulting mixture was stirred at room temperature for 1 hour. After confirming the progress of the reaction by HPLC under the following conditions, H-Glu-[c(RGDfK)]2 (0.50 mg, 0.38 μmol) was added to the reaction solution and allowed to react overnight at room temperature. After confirming the progress of the reaction by HPLC under the same conditions, the reaction solution was diluted with H₂O/MeCN, and the reactant was fractionated by HPLC. After removing MeCN from the collected fraction, a desalination operation was performed. Then the fraction solution was passed through a Na cation exchange resin. The obtained solution was freeze dried to obtain the compound 22 as a blue solid (1.00 mg, 58% yield). The product was identified by HRMS, and the purity was confirmed by analytical HPLC.

HPLC Conditions

Solution A: 0.1M TEAA buffer

Solution B: 99% MeCN/1% H₂O

Liquid flow conditions: 0 to 29 minutes (solution B concentration: 10 to 80%, gradient)

Elution time: 18 minutes

HRMS (ESI⁺): calcd. for C₁₁₀H₁₄₄N₂₃Na₃O₂₃S₂ [M+2H]²⁺1143.9965, found: 1143.9965.

### <Fluorescence Imaging in Cells>

The obtained peptide conjugate was introduced into cultured cells and fluorescence imaging was performed. Human glioblastoma-derived U87MG cells were used as the cultured cells. The U87MG cells are cells expressing high levels of αγβ3. Introduction of the peptide conjugate into the cultured cells was performed in the same manner as in Example 2. When the cells after the introduction of the peptide conjugate were observed with a fluorescence microscope, in both cases of the ICG derivative RGD2-PBA-Cy and the ICG derivative RGD2-PPA-Cy, fluorescence signals were observed from within the cells after culturing for 24 hours following introduction into the cells.

### Example 5

The ICG derivative RGD2-PPA-Cy was administered to cancer cell-transplanted mice to perform fluorescence imaging, and the localization of the ICG derivative RGD2-PPA-Cy within the individual mouse was examined.

All mice (BALB/c, female, 6 to 10 weeks old) were raised in an SPF environment. Animal experiments were conducted in accordance with Hokkaido University Regulations on Animal Experimentation.

First, U87MG cells (5×10⁶ cells/mouse) were subcutaneously transplanted into 6-week-old BALB/c mice (female) to produce cancer cell-transplanted mice. Fourteen days after the transplantation of U87MG cells, mice were administered with the ICG derivative RGD2-PPA-Cy (2 nmol) through the tail vein while anesthetized under isoflurane. Up to 24 hours after administration, the intensity of ICG fluorescence throughout the entire mouse body was measured over time. The intensity of ICG fluorescence was imaged and measured using an imaging system (IVIS Lumina system, manufactured by PerkinElmer Inc.). Excitation light of 740 nm or 780 nm was irradiated and the intensity of fluorescence at 845 nm was measured. Images were processed with “Living Image software v.4.3” (64-bit, manufactured by Caliper Life Sciences, Inc.).

1. An indocyanine green derivative comprising a structure represented by the following general formulas (1) to (3), 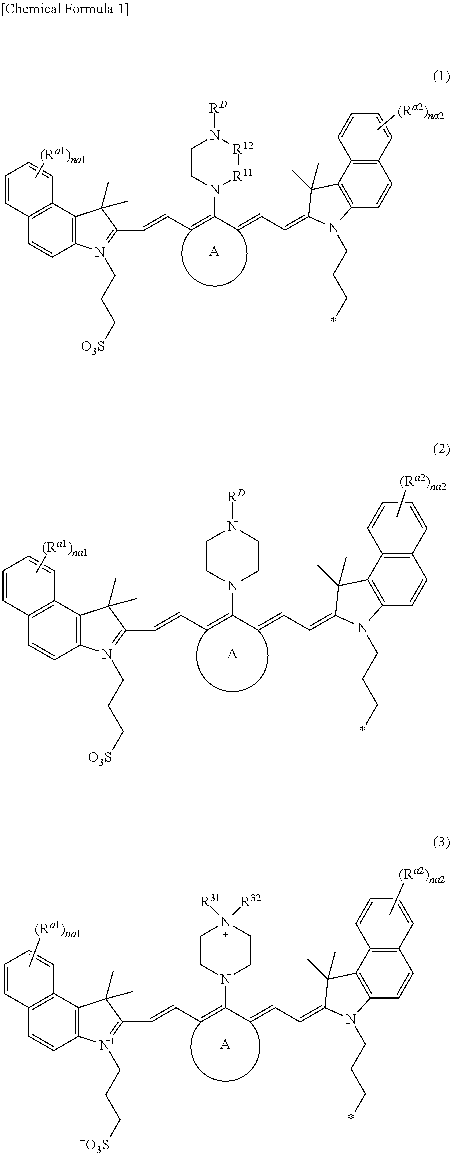
wherein a ring A represents a cyclohexene ring or a cyclopentene ring; R^{D} represents an electron withdrawing group; each of R¹¹ and R¹² independently represents a hydrogen atom or an alkyl group having 1 to 6 carbon atoms which may have a substituent; each of R³¹ and R³² independently represents an alkyl group having 1 to 6 carbon atoms which may have a substituent; each of R^{a1} and R^{a2} independently represents a sulfo group or a carboxy group; each of nₐ₁ and nₐ₂ independently represents 0 or 1; and a filled circle represents a bond.

2. The indocyanine green derivative according to claim 1,
wherein in said general formula (1) or (2), a nitrogen atom bonded to said R^{D} has a stronger electron withdrawing property than that of a nitrogen atom bonded to said ring A.

3. The indocyanine green derivative according to claim 1, comprising a structure represented by said general formula (2),
wherein a distance between a nitrogen atom bonded to said ring A and a carbon atom in said ring A bonded to the nitrogen atom is longer than a distance between a nitrogen atom bonded to a cyclohexene ring in a compound represented by the following formula (Mor-Cy) 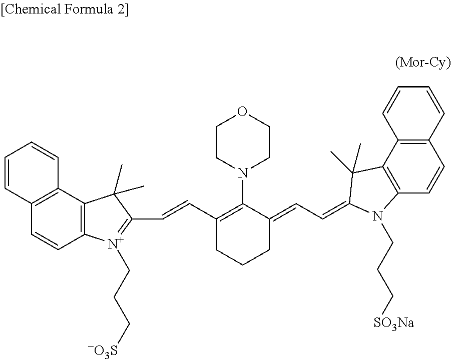
and a carbon atom in said cyclohexene ring bonded to the nitrogen atom, or
an amount of negative charge of the nitrogen atom bonded to said ring A is greater than an amount of negative charge of the nitrogen atom bonded to the cyclohexene ring in the compound represented by said formula (Mor-Cy).

4. The indocyanine green derivative according to claim 1, comprising a structure represented by said general formula (2),
wherein a distance between a nitrogen atom bonded to said ring A and a carbon atom in said ring A bonded to the nitrogen atom is longer than a distance between a nitrogen atom bonded to a cyclohexene ring in a compound represented by the following formula (PhP-Cy) 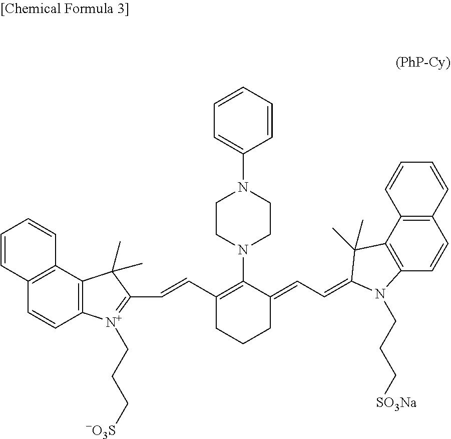
and a carbon atom in said cyclohexene ring bonded to the nitrogen atom, or
an amount of negative charge of the nitrogen atom bonded to said ring A is greater than an amount of negative charge of the nitrogen atom bonded to the cyclohexene ring in the compound represented by said formula (PhP-Cy).

5. The indocyanine green derivative according to claim 1, comprising a structure represented by said general formula (2),
wherein a distance between a nitrogen atom bonded to said ring A and a carbon atom in said ring A bonded to the nitrogen atom is 1.375 Å or more.

6. The indocyanine green derivative according to claim 1, comprising a structure represented by said general formula (2),
wherein an amount of charge of a nitrogen atom bonded to said ring A is −0.524 or less.

7. The indocyanine green derivative according to claim 1,
wherein said R^{D} is an acyl group, a mesyl group, an aldehyde group, a cyano group, a cyanophenyl group, a nitrophenyl group, or a carboxyalkyl group.

8. The indocyanine green derivative according to claim 1,
wherein one or more members selected from the group consisting of a protein, a peptide, a nucleic acid, a sugar, a lipid, a polymer and a low molecular weight compound are linked.

9. The indocyanine green derivative according to claim 8, wherein said protein is an antibody or a portion thereof.

10. A photoacoustic imaging agent comprising the indocyanine green derivative according to claim 1 as an active ingredient.

11. A pharmaceutical composition comprising the indocyanine green derivative according to claim 1 as an active ingredient.

12. A method for producing a photoacoustic imaging image,
the method comprising:
administering the photoacoustic imaging agent according to claim 10 to an individual animal (excluding humans);
irradiating near infrared light from the outside; and
detecting generated photoacoustic waves to produce a photoacoustic imaging image.

13. The method for producing a photoacoustic imaging image according to claim 12, further comprising:
irradiating said individual animal with ultrasonic waves from the outside to produce an echo image; and
superimposing said echo image and said photoacoustic imaging image.
